(19) United States
(12) Patent Application Publication
(10) Pub. No.: US 2022/0273702 A1
(43) Pub. Date: Sep. 1, 2022

(54) HYDROGEN SULFIDE AND NITRIC OXIDE THERAPY FOR COVID-19 INFECTION

(71) Applicant: Board of Supervisors of Louisiana State University and Agricultural and Mechanical College, Baton Rouge, LA (US)

(72) Inventors: Gopi K. Kolluru, Shreveport, LA (US); Christopher KEVIL, Shreveport, LA (US); Paari DOMINIC, Shreveport, LA (US); Anthony Wayne ORR, Benton, LA (US)

(73) Assignee: Board of Supervisors of Louisiana State University and Agricultural and Mechanical College, Baton Rouge, LA (US)

(21) Appl. No.: 17/681,705

(22) Filed: Feb. 25, 2022

Related U.S. Application Data
(60) Provisional application No. 63/153,807, filed on Feb. 25, 2021.

(51) Int. Cl. A61K 33/04; A61K 33/00; A61K 31/715; G01N 33/84

(52) U.S. Cl. CPC A61K 33/04; A61K 33/00; A61K 31/715; G01N 33/84; G01N 2800/26

(57) ABSTRACT

Therapeutics and methods of treating COVID-19 comprising in a patient comprising administering to the patient an effective dose of a pharmacologic composition containing a first therapeutic; wherein the first therapeutic is a hydrogen sulfide (H2S) donor, or a salt, solvate, ester, amide, clathrate, stereoisomer, enantiomer, prodrug or analogs thereof. According to a further embodiment the H2S donor is one of include diallyl trisulfide (DATS), diallyl disulfide (DADS), sodium sulfide, acillin, sugammadex, sulfanilamide, disulfram, sulfonamide, a sulfinate, a sulfoxide, a persulfide, a polysulfide, and a sulfone. According to a further embodiment the pharmacologic composition further contains a second therapeutic, wherein the second therapeutic is a nitrite, or a salt, solvate, ester, amide, clathrate, stereoisomer, enantiomer, prodrug or analogs thereof. According to a further embodiment wherein the nitrite is an inorganic nitrite.

### CROSS REFERENCE TO RELATED APPLICATIONS/PRIORITY

The present invention claims priority to U.S. Provisional Patent Application No. 63/153,807 filed Feb. 25, 2021, which is incorporated by reference into the present disclosure as if fully restated herein. Any conflict between the incorporated material and the specific teachings of this disclosure shall be resolved in favor of the latter. Likewise, any conflict between an art-understood definition of a word or phrase and a definition of the word or phrase as specifically taught in this disclosure shall be resolved in favor of the latter.

### BACKGROUND

Viral illness secondary to coronavirus disease 2019 caused by severe acute respiratory syndrome coronavirus-2 (SARS-CoV-2) is unlike any other previously reported viral diseases, including SARS-CoV due to multiple reasons. First, while the SARS outbreak in 2003 infected approximately 8500 people; COVID-19 as of today has infected hundreds of millions worldwide. Second, the timing with regards to the spread of the virus. In comparison, Flu and SARS-CoV spreads once the infected person exhibits symptoms compared to COVID-19 that can spread up to 3 days prior to symptom development. Third, SARS-CoV-2 is multifold more deadly than other respiratory viruses. Compared to a 0.1-0.2% death rate with Flu infection, COVID-19 has a mortality rate of 2.2% and can be as high as 7% in some countries. COVID-19, unlike other respiratory viruses, causes significant cardiovascular morbidity including myocardial infarction, myocarditis, and cardiac arrhythmias. Interestingly, a large proportion of patients that were asymptomatic or only had mild symptoms with COVID-19 are developing evidence of myocardial involvement and damage, month later that have been confirmed by cardiac MRI imaging. Most importantly a large percentage of these patients are healthy and even athletic individuals, findings that are unique to COVID-19. Finally, COVID-19 long haulers, a large subset of patients that have persistent symptoms for up to 8 months post viral illness is almost unheard of in other viral respiratory illnesses. Therefore, it is of utmost importance to consider therapies for COVID-19 illness as its own entity, notwithstanding previous descriptions in other viral illnesses or other cardiovascular diseases.

### SUMMARY

Wherefore, it is an object of the present invention to overcome the above-mentioned shortcomings and drawbacks associated with the current technology.

The presently disclosed invention relates to therapeutics and methods of treating COVID-19 comprising in a patient comprising administering to the patient an effective dose of a pharmacologic composition containing a first therapeutic; wherein the first therapeutic is a hydrogen sulfide (H2S) donor, or a salt, solvate, ester, amide, clathrate, stereoisomer, enantiomer, prodrug or analogs thereof. According to a further embodiment the H2S donor is one of include diallyl trisulfide (DATS), diallyl disulfide (DADS), sodium sulfide, acillin, sugammadex, sulfanilamide, disulfram, sulfonamide, a sulfinate, a sulfoxide, a persulfide, a polysulfide, and a sulfone. According to a further embodiment the pharmacologic composition further contains a second therapeutic, wherein the second therapeutic is a nitrite, or a salt, solvate, ester, amide, clathrate, stereoisomer, enantiomer, prodrug or analogs thereof. According to a further embodiment wherein the nitrite is an inorganic nitrite. According to a further embodiment the inorganic nitrite is one of sodium nitrite (NaNO₂), ammonium nitrite (NH₄NO₂), barium nitrite (Ba(NO₂)₂; e.g., anhydrous barium nitrite or barium nitrite monohydrate), calcium nitrite (Ca(NO₂)₂; e.g., anhydrous calcium nitrite or calcium nitrite monohydrate), cesium nitrite (CsNO₂), cobalt(II)nitrite (Co(NO₂)₂), cobalt(III)potassium nitrite (CoK₃(NO₂)₆; e.g., cobalt(III)potassium nitrite sesquihydrate), lithium nitrite (LiNO₂; e.g., anhydrous lithium nitrite or lithium nitrite monohydrate), magnesium nitrite (MgNO₂; e.g., magnesium nitrite trihydrate), potassium nitrite (KNO₂), rubidium nitrite (RbNO₂), silver(I)nitrite (AgNO₂), strontium nitrite (Sr(NO₂)₂), and zinc nitrite (Zn(NO₂)₂). According to a further embodiment the inorganic nitrite is NaNO₂.

The presently disclosed invention further relates to kits, therapeutics and methods of diagnosing a severity of a COVID-19 infection in a patient comprising measuring a level of one, two of, or all three of NO metabolites, sulfide metabolites, and nitrotyrosine in a patient; diagnosing the patient with having a severe COVID-19 infection if one of, two of, or all three of the NO metabolite level is low compared to a normal NO metabolite level, the sulfide metabolite level is low compared to a normal sulfide metabolite level, and the nitrotyrosine level is high compared to a normal nitrotyrosine level. According to a further embodiment a plasma sample from the patient is used to test the one of, two of, or all three of the NO metabolite, sulfide metabolite, and nitrotyrosine. According to a further embodiment the sulfide metabolite level includes one, two, or all three of a plasma free sulfide level, an acid labile sulfide level, and a total sulfide level. According to a further embodiment the sulfide metabolite level is not singularly a bound sulfane sulfur level. According to a further embodiment the nitrite metabolite level includes one, two, or all three of a total NO level, a free nitrite level and a S-nitrosothiol (SNO) level. According to a further embodiment a respective level is considered low if it is one of 10% lower, 20% lower, 30% lower, 40% lower, and 50% lower than a respective the normal level, and is considered high if it is one of 10% higher, 20% higher, 30% higher, 40% higher, and 50% higher than the respective normal level. According to a further embodiment, the method further comprises the step of administering to the patient an effective dose of a pharmacologic composition containing a first therapeutic, wherein the first therapeutic is a hydrogen sulfide (H2S) donor, or a salt, solvate, ester, amide, clathrate, stereoisomer, enantiomer, prodrug or analogs thereof, when the patient is diagnosed with having a severe COVID-19 infection. According to a further embodiment the H2S donor is one of include diallyl trisulfide (DATS), diallyl disulfide (DADS), sodium sulfide, acillin, sugammadex, sulfanilamide, disulfram, sulfonamide, a sulfinate, a sulfoxide, a persulfide, a polysulfide, and a sulfone. According to a further embodiment the H2S donor is sugammadex. According to a further embodiment the pharmacologic composition further contains a second therapeutic, wherein the second therapeutic is a nitrite, or a salt, solvate, ester, amide, clathrate, stereoisomer, enantiomer, prodrug or analogs thereof. According to a further embodiment the nitrite is an inorganic nitrite. According to a further embodiment the inorganic nitrite is one of sodium nitrite (NaNO₂), ammonium nitrite (NH₄NO₂), barium nitrite (Ba(NO₂)₂; e.g., anhydrous barium nitrite or barium nitrite monohydrate), calcium nitrite (Ca(NO₂)₂; e.g., anhydrous calcium nitrite or calcium nitrite monohydrate), cesium nitrite (CsNO₂), cobalt(II)nitrite (Co(NO₂)₂), cobalt(III)potassium nitrite (CoK₃(NO₂)₆; e.g., cobalt(III)potassium nitrite sesquihydrate), lithium nitrite (LiNO₂; e.g., anhydrous lithium nitrite or lithium nitrite monohydrate), magnesium nitrite (MgNO₂; e.g., magnesium nitrite trihydrate), potassium nitrite (KNO₂), rubidium nitrite (RbNO₂), silver(I)nitrite (AgNO₂), strontium nitrite (Sr(NO₂)₂), and zinc nitrite (Zn(NO₂)₂).

The presently disclosed invention further relates to kits, devices, chemicals and methods of testing for absence of active COVID-19 infection in a mammal comprising measuring a level of sulfide metabolite in the mammal, and determining an absence of COVID-19 infection in the mammal when the level of sulfide metabolite is equal to or greater than a normal level. According to a further embodiment the sulfide metabolite is free sulfide.

The presently disclosed invention further relates to kits, devices, chemicals and methods of methods of confirming a negative COVID-19 infection test result for a mammal comprising measuring a level of sulfide metabolite in the mammal, and confirming the negative COVID-19 infection test result for the mammal when the level of sulfide metabolite is equal to or greater than a normal level. According to a further embodiment the sulfide metabolite is free sulfide.

The present invention relates to pharmaceutical compositions of a therapeutic (e.g., H2S donors and/or nitrites), or a pharmaceutically acceptable salt, solvate, ester, amide, clathrate, stereoisomer, enantiomer, prodrug or analogs thereof, and use of these compositions for the treatment of a COVID-19 infection and/or the symptoms of C OVID-19 infection.

In some embodiments, the therapeutic, or a pharmaceutically acceptable salt, solvate, or prodrug thereof, is administered as a pharmaceutical composition that further includes a pharmaceutically acceptable excipient.

In some embodiments, administration of the pharmaceutical composition to a human results in a peak plasma concentration of the therapeutic between 0.05 μM-10 μM (e.g., between 0.05 μM-5 μM).

In some embodiments, the peak plasma concentration of the therapeutic is maintained for up to 14 hours. In other embodiments, the peak plasma concentration of the therapeutic is maintained for up to 1 hour.

In some embodiments, the condition is COVID-19.

In certain embodiments, the COVID-19 infection is mild to moderate COVID-19.

In further embodiments, the COVID-19 infection is moderate to severe COVID-19.

In other embodiments, the therapeutic is administered at a dose that is between 0.05 mg-5 mg/kg weight of the human.

In certain embodiments, the pharmaceutical composition is formulated for oral administration.

In other embodiments, the pharmaceutical composition is formulated for extended release.

In still other embodiments, the pharmaceutical composition is formulated for immediate release.

In some embodiments, the pharmaceutical composition is administered concurrently with one or more additional therapeutic agents for the treatment or prevention of COVID-19.

In some embodiments, the therapeutic, or a pharmaceutically acceptable salt, solvate, or prodrug thereof, is administered as a pharmaceutical composition that further includes a pharmaceutically acceptable excipient.

In some embodiments, administration of the pharmaceutical composition to a human results in a peak plasma concentration of the therapeutic between 0.05 μM-10 μM (e.g., between 0.05 μM-5 μM).

In some embodiments, the peak plasma concentration of the therapeutic is maintained for up to 14 hours. In other embodiments, the peak plasma concentration of the therapeutic is maintained for up to 1 hour.

In other embodiments, the therapeutic is administered at a dose that is between 0.05 mg-5 mg/kg weight of the human.

In certain embodiments, the pharmaceutical composition is formulated for oral administration.

In other embodiments, the pharmaceutical composition is formulated for extended release.

In still other embodiments, the pharmaceutical composition is formulated for immediate release.

As used herein, the term “delayed release” includes a pharmaceutical preparation, e.g., an orally administered formulation, which passes through the stomach substantially intact and dissolves in the small and/or large intestine (e.g., the colon). In some embodiments, delayed release of the active agent (e.g., a therapeutic as described herein) results from the use of an enteric coating of an oral medication (e.g., an oral dosage form).

The term an “effective amount” of an agent, as used herein, is that amount sufficient to effect beneficial or desired results, such as clinical results, and, as such, an “effective amount” depends upon the context in which it is being applied.

The terms “extended release” or “sustained release” interchangeably include a drug formulation that provides for gradual release of a drug over an extended period of time, e.g., 6-12 hours or more, compared to an immediate release formulation of the same drug. Preferably, although not necessarily, results in substantially constant blood levels of a drug over an extended time period that are within therapeutic levels and fall within a peak plasma concentration range that is between, for example, 0.05-10 μM, 0.1-10 μM, 0.1-5.0 μM, or 0.1-1 μM.

As used herein, the terms “formulated for enteric release” and “enteric formulation” include pharmaceutical compositions, e.g., oral dosage forms, for oral administration able to provide protection from dissolution in the high acid (low pH) environment of the stomach. Enteric formulations can be obtained by, for example, incorporating into the pharmaceutical composition a polymer resistant to dissolution in gastric juices. In some embodiments, the polymers have an optimum pH for dissolution in the range of approx. 5.0 to 7.0 (“pH sensitive polymers”). Exemplary polymers include methacrylate acid copolymers that are known by the trade name Eudragit® (e.g., Eudragit® L100, Eudragit® S100, Eudragit® L-30D, Eudragit® FS 30D, and Eudragit® L100-55), cellulose acetate phthalate, cellulose acetate trimellitiate, polyvinyl acetate phthalate (e.g., Coateric®), hydroxyethylcellulose phthalate, hydroxypropyl methylcellulose phthalate, or shellac, or an aqueous dispersion thereof. Aqueous dispersions of these polymers include dispersions of cellulose acetate phthalate (Aquateric®) or shellac (e.g., MarCoat 125 and 125N). An enteric formulation reduces the percentage of the administered dose released into the stomach by at least 50%, 60%, 70%, 80%, 90%, 95%, or even 98% in comparison to an immediate release formulation. Where such a polymer coats a tablet or capsule, this coat is also referred to as an “enteric coating.”

The term “immediate release” includes where the agent (e.g., therapeutic), as formulated in a unit dosage form, has a dissolution release profile under in vitro conditions in which at least 55%, 65%, 75%, 85%, or 95% of the agent is released within the first two hours of administration to, e.g., a human. Desirably, the agent formulated in a unit dosage has a dissolution release profile under in vitro conditions in which at least 50%, 65%, 75%, 85%, 90%, or 95% of the agent is released within the first 30 minutes, 45 minutes, or 60 minutes of administration.

The term “pharmaceutical composition,” as used herein, includes a composition containing a compound described herein (e.g., H2S donors and/or nitrites, or any pharmaceutically acceptable salt, solvate, or prodrug thereof), formulated with a pharmaceutically acceptable excipient, and typically manufactured or sold with the approval of a governmental regulatory agency as part of a therapeutic regimen for the treatment of disease in a mammal. Exemplary H2S donors include diallyl trisulfide (DATS), diallyl disulfide (DADS), sodium sulfide, acillin, sugammadex, sulfanilamide, disulfram, sulfonamide, sulfinates, sulfoxides, persulfides, polysulfides, and sulfones. Exemplary nitrites include inorganic nitrites such as sodium nitrite (NaNO₂), ammonium nitrite (NH₄NO₂), barium nitrite (Ba(NO₂)₂; e.g., anhydrous barium nitrite or barium nitrite monohydrate), calcium nitrite (Ca(NO₂)₂; e.g., anhydrous calcium nitrite or calcium nitrite monohydrate), cesium nitrite (CsNO₂), cobalt(II)nitrite (Co(NO₂)₂), cobalt(III)potassium nitrite (CoK₃(NO₂)₆; e.g., cobalt(III)potassium nitrite sesquihydrate), lithium nitrite (LiNO₂; e.g., anhydrous lithium nitrite or lithium nitrite monohydrate), magnesium nitrite (MgNO₂; e.g., magnesium nitrite trihydrate), potassium nitrite (KNO₂), rubidium nitrite (RbNO₂), silver(I)nitrite (AgNO₂), strontium nitrite (Sr(NO₂)₂), and zinc nitrite (Zn(NO₂)₂). It will further be understood that the present invention encompasses all solvated forms (e.g., hydrates) of the nitrite compounds.

Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein.

A “pharmaceutically acceptable excipient,” as used herein, includes any ingredient other than the compounds described herein (for example, a vehicle capable of suspending or dissolving the active compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, or waters of hydration. Exemplary excipients include, but are not limited to: butylated hydroxytoluene (BHT), calcium carbonate, calcium phosphate (dibasic), calcium stearate, croscarmellose, cross-linked polyvinyl pyrrolidone, citric acid, crospovidone, cysteine, ethylcellulose, gelatin, hydroxypropyl cellulose, hydroxypropyl methylcellulose, lactose, magnesium stearate, maltitol, maltose, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The term “pharmaceutically acceptable prodrugs” as used herein, includes those prodrugs of the compounds of the present invention which are, within the scope of sound medical judgment, suitable for use in contact with the tissues of humans and animals with undue toxicity, irritation, allergic response, and the like, commensurate with a reasonable benefit/risk ratio, and effective for their intended use, as well as the zwitterionic forms, where possible, of the compounds of the invention.

The term “pharmaceutically acceptable salt,” as use herein, includes those salts which are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds of the invention or separately by reacting the free base group with a suitable organic or inorganic acid. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

The terms “pharmaceutically acceptable solvate” or “solvate,” as used herein, includes a compound of the invention wherein molecules of a suitable solvent are incorporated in the crystal lattice. A suitable solvent is physiologically tolerable at the administered dose. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

The term “prevent,” as used herein, includes prophylactic treatment or treatment that prevents one or more symptoms or conditions of a disease, disorder, or conditions described herein (e.g., COVID-19). Treatment can be initiated, for example, prior to (“pre-exposure prophylaxis”) or following (“post-exposure prophylaxis”) an event that precedes the onset of the disease, disorder, or conditions. Treatment that includes administration of a compound of the invention, or a pharmaceutical composition thereof, can be acute, short-term, or chronic. The doses administered may be varied during the course of preventive treatment.

The term “prodrug,” as used herein, includes compounds which are rapidly transformed in vivo to the parent compound of the above formula. Prodrugs also encompass bioequivalent compounds that, when administered to a human, lead to the in vivo formation of therapeutic. A thorough discussion is provided in T. Higuchi and V. Stella, Pro-drugs as Novel Delivery Systems, Vol. 14 of the A.C.S. Symposium Series, and Edward B. Roche, ed., Bioreversible Carriers in Drug Design, American Pharmaceutical Association and Pergamon Press, 1987, each of which is incorporated herein by reference. Preferably, prodrugs of the compounds of the present invention are pharmaceutically acceptable.

As used herein, and as well understood in the art, “treatment” includes an approach for obtaining beneficial or desired results, such as clinical results. Beneficial or desired results can include, but are not limited to, alleviation or amelioration of one or more symptoms or conditions; diminishment of extent of disease, disorder, or condition; stabilized (i.e. not worsening) state of disease, disorder, or condition; preventing spread of disease, disorder, or condition; delay or slowing the progress of the disease, disorder, or condition; amelioration or palliation of the disease, disorder, or condition; and remission (whether partial or total), whether detectable or undetectable. “Treatment” can also mean prolonging survival as compared to expected survival if not receiving treatment. As used herein, the terms “treating” and “treatment” can also include delaying the onset of, impeding or reversing the progress of, or alleviating either the disease or condition to which the term applies, or one or more symptoms of such disease or condition.

The term “unit dosage forms” includes physically discrete units suitable as unitary dosages for human subjects and other mammals, each unit containing a predetermined quantity of active material calculated to produce the desired therapeutic effect, in association with any suitable pharmaceutical excipient or excipients.

As used herein, the term “plasma concentration” includes the amount of therapeutic present in the plasma of a treated subject (e.g., as measured in a rabbit using an assay described below or in a human).

Various objects, features, aspects, and advantages of the present invention will become more apparent from the following detailed description of preferred embodiments of the invention, along with the accompanying drawings in which like numerals represent like components. The present invention may address one or more of the problems and deficiencies of the current technology discussed above. However, it is contemplated that the invention may prove useful in addressing other problems and deficiencies in a number of technical areas. Therefore, the claimed invention should not necessarily be construed as limited to addressing any of the particular problems or deficiencies discussed herein.

### BRIEF DESCRIPTION OF THE DRAWINGS

The accompanying drawings, which are incorporated in and constitute a part of the specification, illustrate various embodiments of the invention and together with the general description of the invention given above and the detailed description of the drawings given below, serve to explain the principles of the invention. It is to be appreciated that the accompanying drawings are not necessarily to scale since the emphasis is instead placed on illustrating the principles of the invention. The invention will now be described, by way of example, with reference to the accompanying drawings in which:

FIGS. 1A-1C show Plasma NO availability in COVID-19 patients. Results show significantly reduced total NO (FIG. 1A), free nitrite (FIG. 1B), and s-nitrosothiol (FIG. 1C) metabolites in COVID-19 patients (n=68) compared to control subjects (n=33.

FIGS. 2A-2D show plasma sulfide pools in COVID-19 patients. Scatter bar graphs showing plasma free sulfide (FIG. 2A), acid labile sulfide (FIG. 2B), bound sulfane sulfur (FIG. 2C) and total sulfides (FIG. 2D) in Control and COVID-19 subjects. Results show significantly reduced sulfide metabolites with the exception of bound sulfane sulfur in COVID-19 patients (n=68) compared to Controls (n=33).

FIGS. 3A-3F show NO availability by race. Total NO, free nitrite, and s-nitrosothiol metabolites are significantly reduced in Caucasian (FIGS. 3A-3C) COVID patients (n=21) compared to controls (n=19). There was a trend towards lower free nitrite levels and significantly reduced total NO and s-nitrosothiol metabolites in African American (FIGS. 3D-3F) COVID-19 patients (n=44) compared to control subjects (n=13).

FIGS. 4A-4H show sulfide pools by race. Free sulfide, acid labile sulfide, bound sulfane sulfur and total sulfides in Caucasian (FIGS. 4A-4D) and African American (FIGS. 4E-4H) COVID-19 subjects compared to control subjects, respectively. Scatter bar graphs show a significantly reduced total and free sulfide levels but not bound sulfane sulfur and acid labile sulfide levels in Caucasian COVID-19 patients (n=18) compared to controls (n=19); and significantly reduced total, free and acid labile sulfide levels but comparable bound sulfane sulfur levels in African American COVID-19 patients (n=46) compared to controls (n=13).

FIGS. 5A-5C show nitrotyrosine levels in controls vs COVID-19 patients. Nitrotyrosine levels are significantly increased in COVID-19 patients (n=68) compared to healthy controls (n=33) in the overall study population (FIG. 5A); There was a similar increase in nitrotyrosine levels in the Caucasian (n=21 vs 19) (FIG. 5B) and African American (n=44 vs 13) (FIG. 5C) COVID patients compared to race matched controls.

FIG. 6 shows a single case of COVID-19 infection and its association with CRP, NO and sulfide levels. Nitrotyrosine levels of the subject pre-COVID, during and post-COVID at 5 days and 14 days (top panel); CRP levels during and post-COVID, 3 days and 5 days (middle panel); NO and sulfide levels before, during and post-COVID at 5 days and 14 days (bottom panel).

FIGS. 7A-7F show receiver-operating characteristic analysis (ROC) of NO metabolites in controls vs COVID. ROC curves with area under the curve of Total NO and S-nitrosothiol—altogether (FIGS. 7A and 7B); Caucasians (FIGS. 7C and 7D) and African American (FIGS. 7E and 7F) populations, respectively.

FIGS. 8A-8F show receiver-operating characteristic analysis (ROC) of Sulfide in controls vs COVID. ROC curves with area under the curve of Free and total sulfides of COVID-19 subjects altogether (FIGS. 8A and 8B); Caucasian population (FIGS. 8C and 8D); and African American populations (FIGS. 8E and 8F) respectively.

FIGS. 9A-9C show receiver-operating characteristic analysis (ROC) of NO metabolites in controls vs COVID. ROC curves with area under the curve of free nitrite—altogether (FIG. 9A); Caucasians (FIG. 9B) and African American (FIG. 9C) populations, respectively.

FIGS. 10A-10F show receiver-operating characteristic analysis (ROC) of Sulfide in controls vs COVID. ROC curves with area under the curve of free and total sulfides of COVID-19 subjects altogether (FIGS. 10A and 10B); Caucasian population (FIGS. 10C and 10D); and African American populations (FIGS. 10E and 10F) respectively.

FIGS. 11A and 11B show a Replication-Competent, Infectious VSV Chimera with SARS-CoV-2 S Protein (VSV-eGFP-SARS-CoV-2-SAA). We have infected Vero E6 cells with VSV-eGFPSARS-CoV-2-SAA (simply SARS-CoV-2, FIG. 11A) as determined by expression of the virus-encoded eGFP reporter (FIG. 11B).

FIGS. 12A-12C show Representative western blots from Control (Vero E6 cells alone); cells infected for 48 hrs with SARS-CoV2 and 50 mM DATS and cells+SARS-CoV2 (FIG. 12A). Quantification of CSE and phospho-eNOS respectively (FIGS. 12B and 12C).

### DETAILED DESCRIPTION

The present invention will be understood by reference to the following detailed description, which should be read in conjunction with the appended drawings. It is to be appreciated that the following detailed description of various embodiments is by way of example only and is not meant to limit, in any way, the scope of the present invention. In the summary above, in the following detailed description, in the claims below, and in the accompanying drawings, reference is made to particular features (including method steps) of the present invention. It is to be understood that the disclosure of the invention in this specification includes all possible combinations of such particular features, not just those explicitly described. For example, where a particular feature is disclosed in the context of a particular aspect or embodiment of the invention or a particular claim, that feature can also be used, to the extent possible, in combination with and/or in the context of other particular aspects and embodiments of the invention, and in the invention generally. The terms “comprise(s),” “include(s),” “having,” “has,” “can,” “contain(s),” and grammatical equivalents and variants thereof, as used herein, are intended to be open-ended transitional phrases, terms, or words that do not preclude the possibility of additional acts or structures. are used herein to mean that other components, ingredients, steps, etc. are optionally present. For example, an article “comprising” (or “which comprises”) components A, B, and C can consist of (i.e., contain only) components A, B, and C, or can contain not only components A, B, and C but also one or more other components. The singular forms “a,” “and” and “the” include plural references unless the context clearly dictates otherwise. Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range beginning with that number (which may be a range having an upper limit or no upper limit, depending on the variable being defined). For example, “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40% means 40% or less than 40%. When, in this specification, a range is given as “(a first number) to (a second number)” or “(a first number)-(a second number),” this means a range whose lower limit is the first number and whose upper limit is the second number. For example, 25 to 100 mm means a range whose lower limit is 25 mm, and whose upper limit is 100 mm.

The embodiments set forth the below represent the necessary information to enable those skilled in the art to practice the invention and illustrate the best mode of practicing the invention. For the measurements listed, embodiments including measurements plus or minus the measurement times 5%, 10%, 20%, 50% and 75% are also contemplated. For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “substantially” means that the property is within 80% of its desired value. In other embodiments, “substantially” means that the property is within 90% of its desired value. In other embodiments, “substantially” means that the property is within 95% of its desired value. In other embodiments, “substantially” means that the property is within 99% of its desired value. For example, the term “substantially complete” means that a process is at least 80% complete, for example. In other embodiments, the term “substantially complete” means that a process is at least 90% complete, for example. In other embodiments, the term “substantially complete” means that a process is at least 95% complete, for example. In other embodiments, the term “substantially complete” means that a process is at least 99% complete, for example.

The term “substantially” includes a value is within about 10% of the indicated value. In certain embodiments, the value is within about 5% of the indicated value. In certain embodiments, the value is within about 2.5% of the indicated value. In certain embodiments, the value is within about 1% of the indicated value. In certain embodiments, the value is within about 0.5% of the indicated value.

The term “about” includes when value is within about 10% of the indicated value. In certain embodiments, the value is within about 5% of the indicated value. In certain embodiments, the value is within about 2.5% of the indicated value. In certain embodiments, the value is within about 1% of the indicated value. In certain embodiments, the value is within about 0.5% of the indicated value.

In addition, the invention does not require that all the advantageous features and all the advantages of any of the embodiments need to be incorporated into every embodiment of the invention.

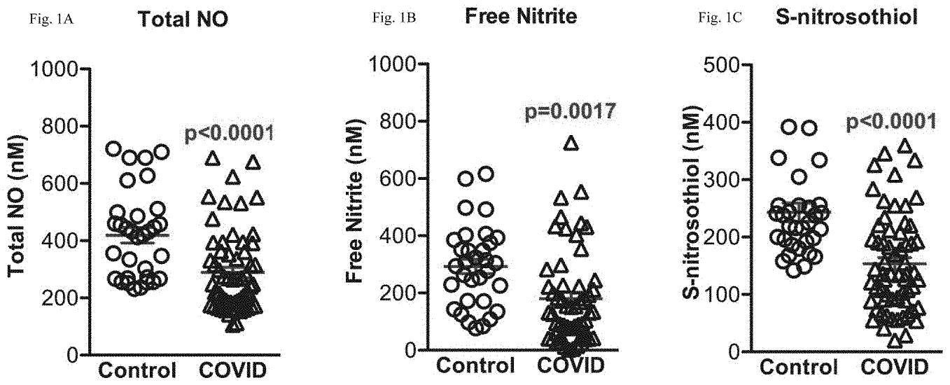

Turning now to FIGS. 1A-12C, a brief description concerning the various components of the present invention will now be briefly discussed. Coronavirus disease 2019 (COVID-19) caused by severe acute respiratory syndrome coronavirus 2 (SARS-CoV-2) has infected over 400 million people in over 220 countries during the recent worldwide pandemic, approximately 78 million of whom are in the United States. Although COVID-19 causes significant morbidity and mortality when it manifests as ‘viral pneumonia,’ available evidence suggests that COVID-19 is associated with cardiovascular complications. These are rapidly emerging as a key threat, leading to increasing hospitalizations accompanied by a host of complications, including myocarditis, thrombo-embolism, acute coronary syndrome, and resultant cardiac arrhythmias, together referred to as Acute COVID-19 Cardiovascular Syndrome (ACovCS. The complications of COVID-19 are significantly exacerbated due to preexisting comorbidities, including pulmonary and cardiovascular disease. Studies of the SARS and SARS-CoV-2 viruses reveal a potential role for cytokine storm, altered blood pressure regulation, and thrombosis in the pathogenesis of COVID-19. Moreover, COVID-19 has been shown to directly target endothelial cells and cause endotheliitis, thus affecting downstream functions that may contribute to cardiovascular complications. However, the link between cardiovascular complications and COVID-19, along with the underlying molecular mechanisms, remains poorly understood.

Nitric oxide (NO) and hydrogen sulfide (H₂S) are ubiquitous signaling molecules popularly referred to as gasotransmitters that play protective roles in limiting the severity of cardiovascular disease. NO acts as a vasodilator and an antiviral agent in patients with SARS and can inhibit in vitro replication of SARS-CoV-2. While several recent reviews also suggest an association between H₂S and SARS-CoV-1/2, they provide little evidence of any of such relationship. Consistent with these suppositions is the possibility that endothelial dysfunction concomitant with COVID-19 infection is likely to result in reduced NO and H₂S metabolite availability. However, no studies have been reported to date evaluating specific levels of gasotransmitters in relation to COVID-19. In this study, we assessed the relationship between NO and H₂S metabolite availability in patients with COVID-19 and further evaluated them as prognostic biomarkers in severely ill COVID-19 patients.

Methods. Study design. This was a case-control study approved by the Institutional Review Board (IRB) of Louisiana State University Health Sciences Center at Shreveport (LSUHSC-S) (STUDY00001501). Consecutive patients admitted with COVID-19 viral pneumonia to Ochsner-LSU hospital in Shreveport were approached for inclusion in the study. Patients who tested positive for COVID-19 by rapid testing or by PCR within 14 days were included. Pregnant women, prisoners, and patients younger than 18 years of age or older than 89 years of age were excluded from the study. Among those who met the inclusion criteria, a total of 73 patients were consented; two patients withdrew their consent, we could not obtain blood samples from two other patients, and one sample was inadequate for performing analysis. Volunteers were invited to enroll in the study using flyers and by word of mouth. Blood samples from healthy race- and sex-matched volunteers with no prior history of COVID-19 infection were also obtained in the cardiology clinic at Ochsner-LSU Hospital in Shreveport after the volunteers provided an informed consent.

Human blood collection. After obtaining an informed consent, blood samples were collected from human healthy subjects and COVID-19 patients into 6 mL BD vacutainer tubes with lithium heparin. Samples were transported to the lab within 15 min on ice and were centrifuged at 1500 RCF for 4 min at 4 C; plasma was collected and snap frozen for further analyses. Medical record data pertaining to baseline characteristics and comorbidities of healthy subjects and COVID-19 patients were collected and compared (Table 1, Table 2).

TABLE 1
Demographics of COVID-19 cases and healthy controls included in the study.

| | MA | N | C | AA | OR | M | | F | |
|---|---|---|---|---|---|---|---|---|---|
| Controls | 43.09 (22-68) | 33 | 19 (58%) | 13 (39%) | 1 (3%) | 18 | (54%) | 15 | (45%) |
| C19 P | 58.19 (27-85) | 68 | 21 (31%) | 45 (66%) | 2 (3%) | 35 | (51.5%) | 33 | (48.5%) |

MA = mean age (range);
N = total;
C = Caucasians;
AA = African Americans;
OR = Other Race;
M = males;
F = females;
C19 P = Covid-19 Patients

TABLE 2
Patient and disease characteristics of
COVID-19 cases included in the study.

| | | | African | | | |
|---|---|---|---|---|---|---|
| Patient | Total Number | | Americans | | Caucasians | |
| Characteristics | (% of Total Number) | | (AA) | | (C) | |
| Comorbidities | | | | | | |
| DM | 29/68 | (42.6%) | 21/45 | (46.7%) | 6/21 | (28.5%) |
| Hypertension | 51/68 | (75%) | 39/45 | (86.6%) | 11/21 | (52.4%) |
| BMI > 30 | 41/68 | (60%) | 27/45 | (60%) | 12/21 | (57.1%) |
| COPD | 6/68 | (8.8%) | 5/45 | (11.1%) | 1/21 | (4.7%) |
| CVD | 18/68 | (26.4%) | 12/45 | (26.7%) | 6/21 | (28.6%) |
| Severity | | | | | | |
| Mild-Moderate | 46/68 | (67.6%) | 33/45 | (73.3%) | 12/21 | (57.1%) |
| Severe | 22/68 | (32.3%) | 12/45 | (26.7%) | 9/21 | (42.8%) |

DM = Diabetes Mellitus; BMI = Body Mass Index; COPD = chronic obstructive pulmonary disease; CVD = cardiovascular disease. Two patients in the study were Hispanic.

NO metabolite measurements. NO metabolites (NOx) were measured using an ozone-based chemiluminescence assay (Sievers Nitric Oxide Analyzer 280i, Weddington, N.C.) as described previously. Plasma samples were collected in NO stabilization buffer (1.25 mol/L potassium ferricyanide, 56.9 mmol/L N-ethylmaleimide, 6% Nonidet P-40 substitute in PBS), or free nitrite and S-nitrosothiol (SNO) preservation buffers (Zysense, Weddington, N.C.), respectively. Aliquots of samples were injected into the analyzer and tested for total NO and for individual NO metabolites.

Measurement of biological pools of H₂S. Plasma samples were analyzed for free sulfide, acid-labile sulfide (ALS), bound sulfane sulfur (BSS), and total sulfide levels using the monobromobimane (MBB) method reported previously. Free sulfide was measured using 30 μL of plasma with MBB; for detection of ALS and BSS, 50 μL of plasma was processed separately in two 4 mL BD vacutainer tubes with 100 mM phosphate buffer (pH 2.6, 0.1 mM DTPA) for the ALS reaction, and 100 mM phosphate buffer (pH 2.6, 0.1 mM DTPA) containing 1 mM TCEP for the total sulfide reaction. Following a 30-minute incubation on a nutator mixer, to trap the evolved sulfide gas and incubation with 100 mM Tris-HCl buffer (pH 9.5, 0.1 mM DTPA) for 30 minutes on a nutator mixer, the trapped sulfide was then measured using the MBB method and calculations performed to determine total sulfide and its pools as previously described

Measurement of nitrotyrosine. Quantitative determination of nitrotyrosine in the plasma of control subjects and COVID-19 patients was performed by a competitive ELISA kit (Cell biolabs, Inc.) as per manufacturer's instructions.

Statistical analyses. Levels of NO and sulfide metabolites were assessed by group means and standard deviations with subsequent pairwise comparison using analysis of variance (ANOVA). Receiver-operating characteristic analysis (ROC) was conducted to assess the predictive accuracy in correlating NO and sulfide levels with COVID-19 infection. Cutoff values for positive classification were included in the curve, with a nonparametric distribution assumption and a confidence level of 95%. These statistical analyses were performed using GraphPad Prism 5.0. We also conducted multivariable regression analyses to estimate the effect of various predictor variables on NO and H₂S in separate models with 95% confidence intervals. A descriptive analysis of study variables was performed using SPSS Version 26.0 (IBM Corp., Armonk, N.Y.). A Chi-square test of independence was used to determine associations between categorical variables. For continuous variables, means of two independent groups were compared using the independent samples Student's t-test. For all analyses, a p-value of <0.05 was considered statistically significant. We assumed equal variance for the independent samples Student's t-test result when Levene's test had a p-value <0.05. Otherwise, we used the results from equal variance not assumed. For the purposes of this disclosure, the various measurements of levels present in the controls is considered a normal level for the respective values measured.

Results. NO metabolites are reduced with COVID-19 infection. A total of 68 COVID-19 cases and 33 controls were included in the study. Plasma NO availability was measured and compared between control subjects and COVID-19 patients (FIG. 1). We found a significant reduction in the total NO levels in the plasma of COVID-19 patients compared to that of healthy controls (FIG. 1A; 418.84±153.03 nM vs 286.69±140.39 nM, p<0.0001). In addition, to observe the effect of COVID-19 infection on individual NO metabolites, we measured free nitrite (FIG. 1B) and bound SNO fractions (FIG. 1C) using commercially available stabilization buffers. Free nitrite (292.63±141.67 nM vs 179.945±164.0 nM, p=0.0017) and SNO fractions (243.19±91.60 nM vs 152.89±85.39 nM, p<0.0001) were significantly reduced in the plasma of COVID-19 patients compared to that of the controls (FIGS. 1B and C).

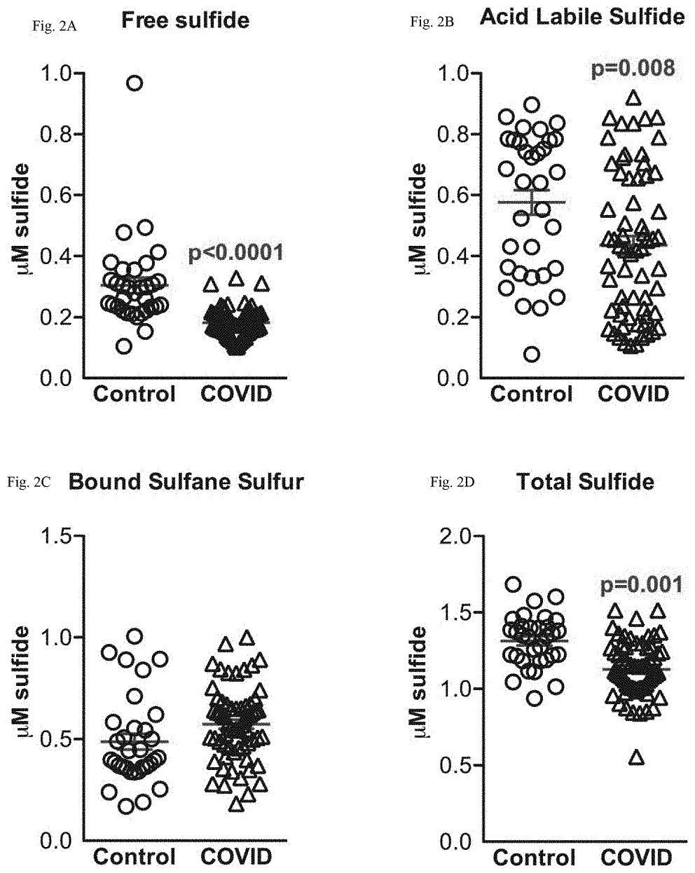

Sulfide pools are reduced with COVID-19 infection. We next examined the impact of COVID-19 infection on sulfide metabolites. FIG. 2 illustrates free, acid labile, bound sulfide, and total sulfide pools that were quantified in plasma samples from healthy controls and COVID-19 patients. Sulfide levels, including free (0.31±0.14 μM vs 0.18±0.05 μM, p<0.0001; FIG. 2A), ALS (0.59±0.23 μM vs 0.45±0.24 μM, p=0.008; FIG. 2B) and total (1.37±0.31 μM vs 1.15±0.21 μM, p=0.001; FIG. 2D), were significantly reduced in COVID-19 patients compared to the healthy controls. No significant differences were observed in BSS (0.53±0.32 μM vs 0.59±0.19 μM; FIG. 2C).

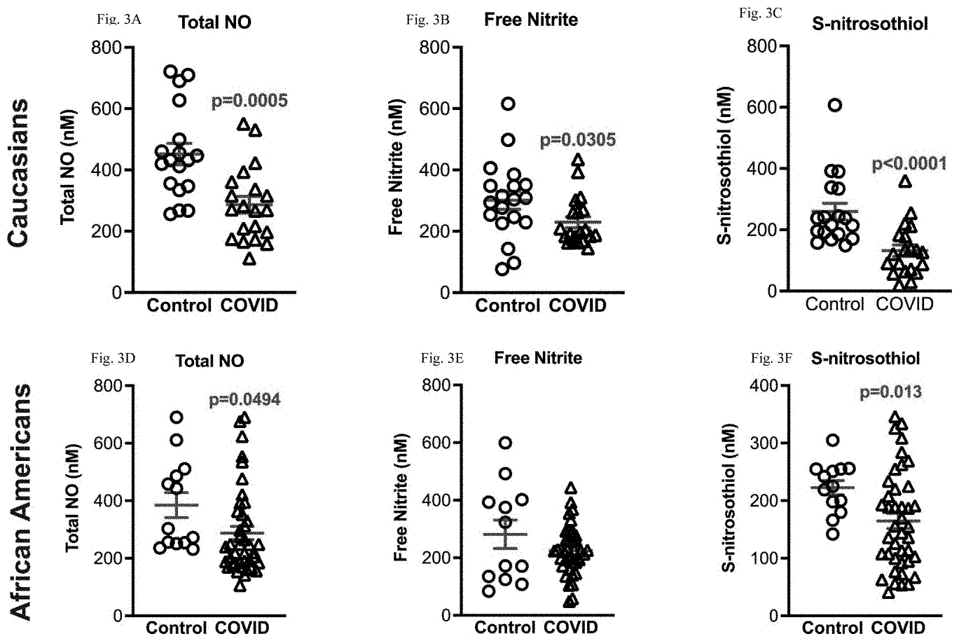

Race-based comparison of NO metabolites in COVID-19 patients. The association of plasma NO levels were compared between COVID-19 patients and control subjects based on race. Analysis by race revealed a significant reduction in plasma total (451.8±158 nM vs 286.35±120.55 nM; p=0.0005), free nitrite (301.16±128.37 nM vs 229.55±79.09 nM; p=0.03), and SNO (259.56±115.10 nM vs 131.80±83.98 nM; p<0.0001) metabolites in Caucasian COVID-19 patients compared to race matched controls (FIG. 3A-C), whereas NO metabolites in African Americans (AA) showed a significant reduction in total NO (384.8±157 nM vs 287.6±150.3 nM; p=0.0494) and SNO levels (222.62±44.57 nM vs 164.7±85.06 nM; p=0.013) in COVID-19 patients compared to AA controls (FIGS. 3D and F). Although a trend towards decreased free nitrite (281.58±171.04 nM vs 224.1±85.37 nM; p=0.275) was seen in AA COVID-19 patients compared to AA controls, no statistical significance was observed (FIG. 3E). Moreover, no race-based differences were observed when NO levels were compared between control and/or COVID-19 groups in Caucasians vs AA.

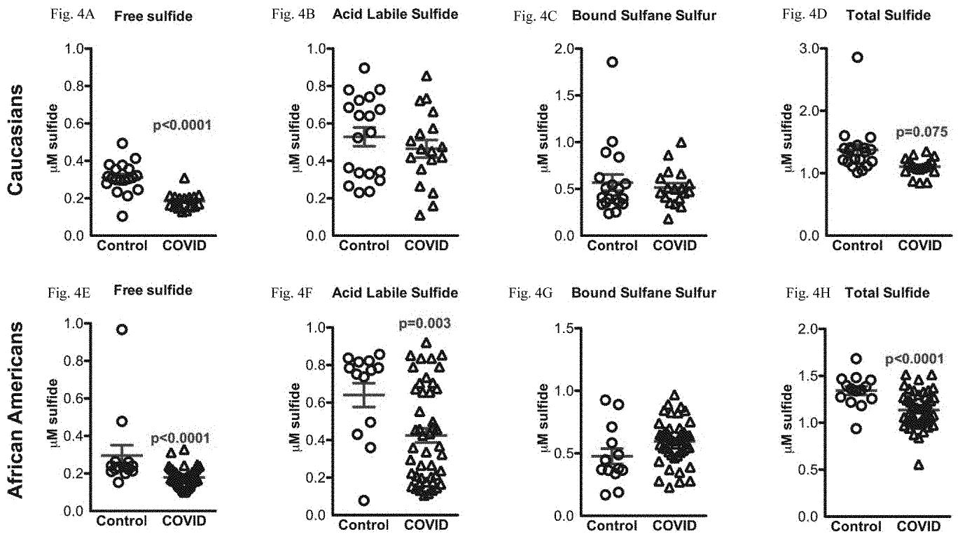

Race-based comparison of sulfide metabolites in COVID-19 patients. We next compared subjects based on race for sulfide metabolites (FIG. 4A-D). A significant reduction was seen in free sulfide pools (0.31±0.08 μM vs 0.19±0.06 μM, p<0.0001) and total sulfide levels (1.37±0.40 μM vs 1.19±0.24 μM, p=0.075) in Caucasian COVID-19 patients compared to healthy subjects (FIGS. 4A and 4D). The reduced levels of ALS and BSS in Caucasian COVID-19 patients were not statistically significant. In the AA population, a significant decrease was seen in free (0.25±0.08 μM vs 0.18±0.05 μM, p<0.0001), acid labile (0.67±0.23 μM vs 0.43±0.250 μM, p=0.003), and total sulfide levels (1.37±0.13 μM vs 1.13±0.19 μM, p<0.0001), while no significant changes were seen in the levels of BSS (FIG. 4G). When sulfide levels were compared between Caucasian and AA controls, there was a significant reduction in free sulfide levels (0.31±0.08 μM vs 0.25±0.08 μM; p=0.04) in AA subjects. No significance was seen in other pools of sulfide in comparisons between these races in either the control or COVID-19 groups.

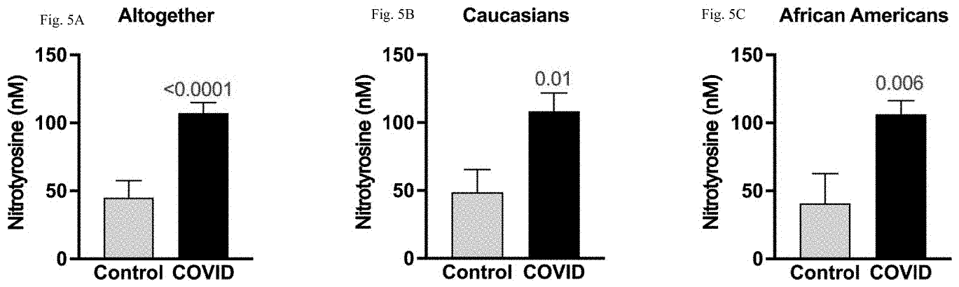

Nitrotyrosine levels are elevated in COVID-19 patients. To determine NO-derived oxidants, we measured systemic levels of nitrotyrosine in the plasma from healthy controls and COVID-19 patients (FIG. 5). Nitrotyrosine levels were significantly higher among patients with COVID-19 compared to healthy controls (107.049±7.907 nM vs 44.7606±12.85 nM; P<0.0001; FIG. 5A). Analysis by race showed a significant increase in nitrotyrosine levels both in Caucasian COVID-19 patients (108.2±13.62 nM vs 48.54±16.92 nM; p=0.01; FIG. 5B), and in African Americans COVID-19 patients (106.2±10.01 nM vs 40.69±22.01 nM; p=0.006; FIG. 5C) compared to respective race matched controls.

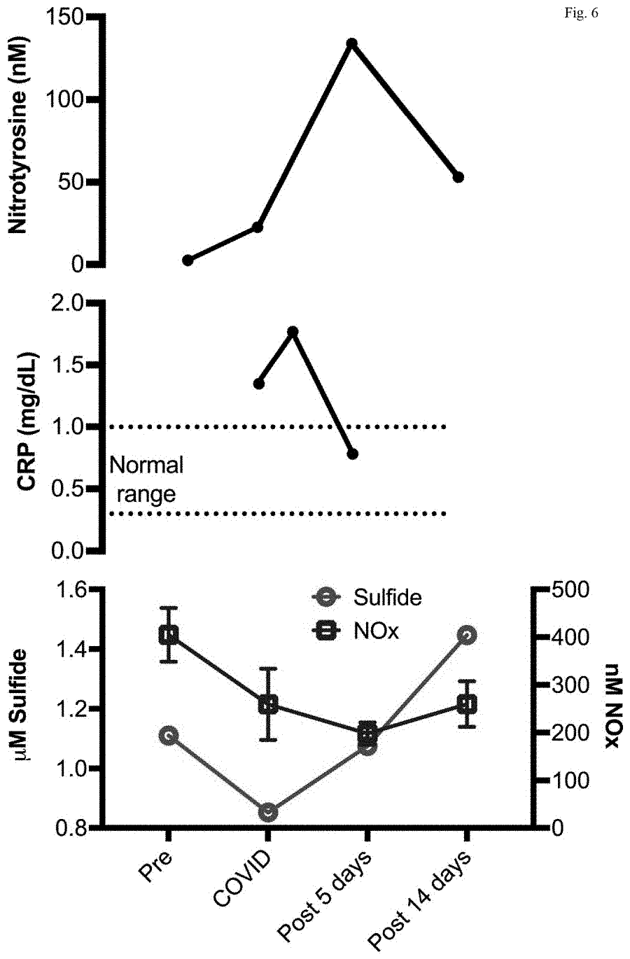

A case-study of a single COVID-19 patient-association between CRP and gasotransmitters. C-reactive protein (CRP) levels have been shown to be an early prognosticator in COVID-19 pneumonia and can indicate disease severity, whereas the gasotransmitters NO and H₂S are known to the inventors for their anti-inflammatory properties. We measured nitrotyrosine, CRP, as well as NO and H₂S levels in a single subject who was initially a control subject, but 9 days later contracted a COVID-19 infection (FIG. 6). The subject's CRP levels, which were significantly elevated with COVID-19 infection (1.35 mg/dL, normal range 0.3-1.0 mg/dL), were further elevated (1.77 mg/dL) within 3 days of infection, and then returned to the normal range (0.78 mg/dL) following antiviral therapy with remdesivir for 5 days (FIG. 6, middle panel). Total NO and sulfide levels were significantly reduced during COVID-19 infection (280 nM and 0.8523 μM, respectively) in this individual from pre-infection baseline (400 nM and 1.11039 μM, respectively) (FIG. 6, bottom panel). However, both the NO and sulfide levels were elevated following remdesivir antiviral therapy coinciding with decreased COVID-19 symptoms (5 days post-treatment: 200 nM and 1.07555 μM; 14 days post-treatment: 280 nM and 1.44706 μM). The subject's level of nitrotyrosine, an oxidant marker, was significantly increased with COVID-19 infection and at day-5 post COVID-19 infection (22.52 nm and 133.99 nM respectively) compared to the baseline (2.56 nM) (FIG. 6, top panel), in close alignment with the increasing CRP levels and decreasing NO and H₂S levels. Nitrotyrosine level then steeply decreased at day-14 post COVID-19 infection (52.97 nM) with corresponding increase in NO and H₂S levels.

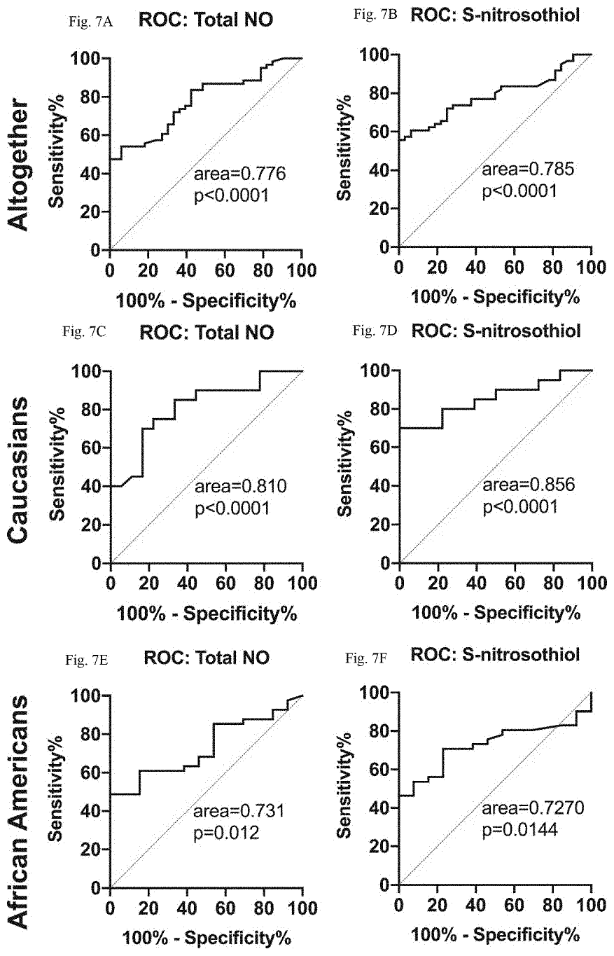

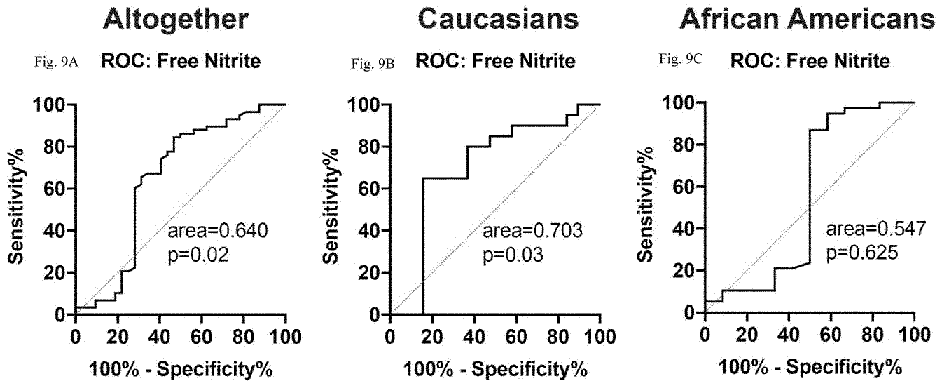

Nitric oxide as an indicator of COVID-19 infection. We performed receiver-operating characteristic analysis (ROC) (FIGS. 7 and 9) to determine the accuracy of reduced NO levels as an indicator of COVID-19. Analysis of plasma NO and its metabolites between COVID-19 patients and controls revealed areas under the curve (AUC) of 0.776 (p<0.0001), 0.640 (p=0.02), and 0.785 (p<0.0001) for total NO, free nitrite, and SNO, respectively (FIGS. 7A, 7B, and 9A). Plasma NO metabolites were then analyzed based on race in Caucasian and AA subjects, and found to be a stronger indicator of COVID-19 infection in Caucasian patients (AUC of 0.810, p<0.0001; 0.703, p=0.03; and 0.856, p<0.0001 (FIGS. 7D, 7E; 9B) for total NO, free nitrite, and SNO, respectively) compared to AA (AUC 0.731, p=0.012 and 0.727, p=0.014 total and SNO, respectively (FIG. 7G, I)). However, free nitrite levels in AA subjects did not show any significant predictability for COVID-19 infection (AUC of 0.547, p=0.625, FIG. 9C).

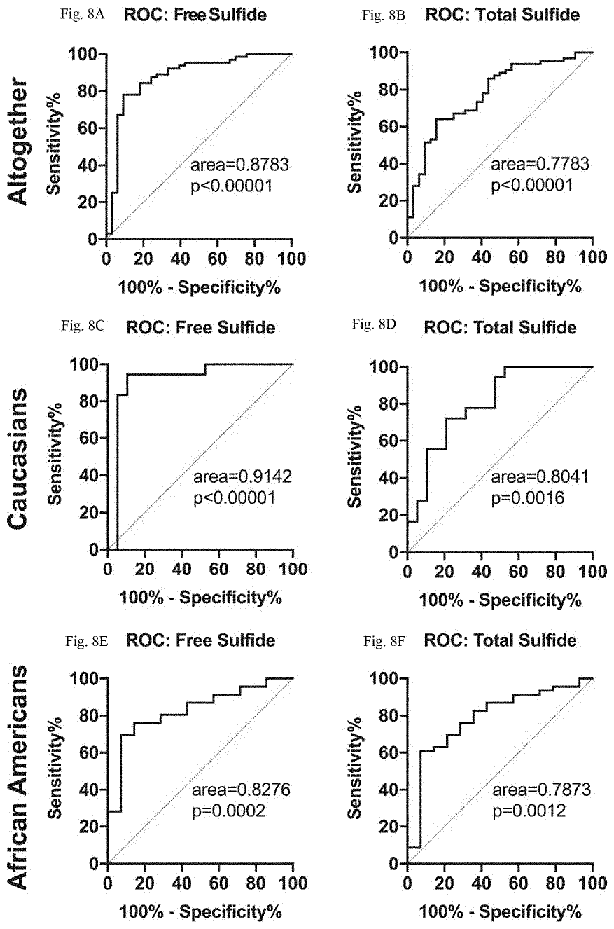

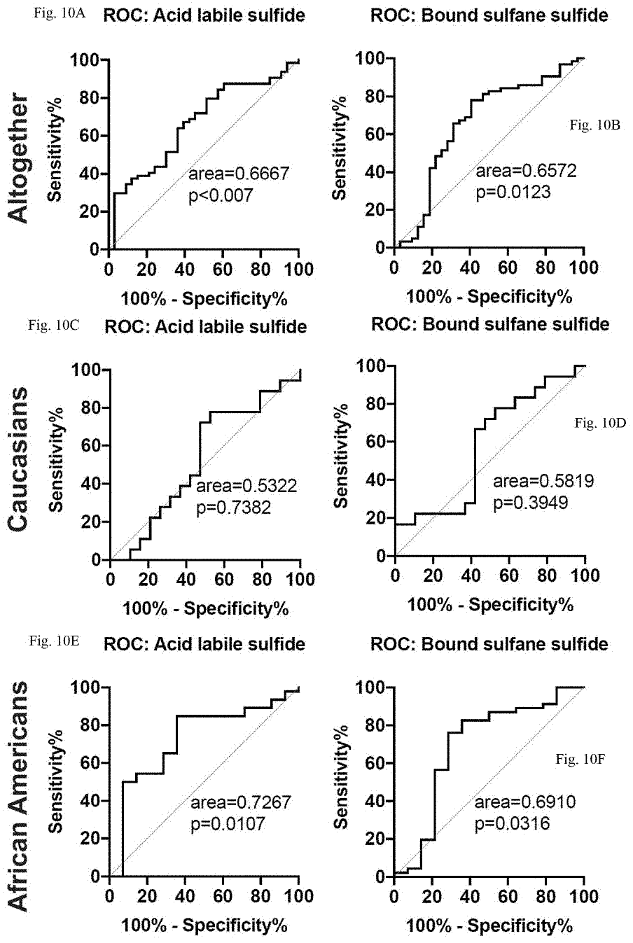

Sulfide pools as indicators of COVID-19 infection. We next performed ROC with sulfide and its metabolites by analyzing the AUC in healthy controls and COVID-19 patients. Free sulfide with an AUC of 0.8697 (95% CI—0.7878-0.9517, p<0.0001) was a strong predictor of COVID-19 in the overall study population (FIG. 8A). A free sulfide of 0.30 μM or below had a sensitivity of 96% and a specificity of 33% of predicting COVID-19 infection and a level of 0.24 μM or below had a sensitivity of 91% with a specificity of 67%. Total sulfide was also fairly able to predict COVID-19 infection with an AUC of 0.753 (p<0.0001, FIG. 8B). We further analyzed the accuracy of reduced sulfide levels as a predictor of COVID-19 based on race in Caucasian and AA subjects. We found that free sulfide was a powerful predictor of COVID-19 infection in Caucasians with an AUC of 0.915 (p<0.0001, FIG. 8C). A free sulfide level of 0.30 μM or less was 94% sensitive and 58% specific in predicting COVID-19 infection in Caucasians. Total sulfide with an AUC of 0.8041 (p=0.0016) in Caucasians was a fair predictor of COVID-19 infection in this population. Total and free sulfide with an AUC of 0.7873 (p<0.0001) and 0.8276 (p<0.0001) respectively were good predictors of COVID-19 infection in AA patients (FIGS. 8E and F). With a free sulfide level of 0.30 μM or below, the sulfide levels were able to predict COVID-19 with 95% sensitivity and 14% specificity in AA. As shown in FIGS. 10A-10F, acid labile sulfide and bound sulfane sulfide were also analyzed, and their sensitivity and specificity are shown.

Correlation and regression analyses between COVID-19 severity and gasotransmitters. Independent sample Student's t-tests were performed to study the association between biomarkers (cardiac injury, thrombosis, and inflammatory) and NO and H₂S levels in the COVID-19 cases (Table 3). LDH levels ≤260 U/L compared to LDH>260 U/L showed significant differences in NO levels (210.50±64.41 nM vs 323.16±167.63 nM; p=0.004) (Table 3). In addition, there was a trend towards a difference in NO levels in patients with low and high levels of lactate (235.65±64.79 nM vs 311.26±177.96 nM, p=0.052) and procalcitonin (251.34±102.73 nM vs 334.70±165.37 nM, p=0.053). Based on the level of respiratory support, COVID-19 patients were categorized as mild-to-moderately ill or severely ill. Compared to patients with mild-to-moderately severe COVID-19 illness, patients with severe illness had slightly elevated NO (274.02±133.67 nM (n=46) versus 314.68±154.25 nM (n=22), p=0.299). Similarly, patients who died had significantly higher levels of NO compared to levels in patients who survived (263.65±124.34 nM (n=60) versus 464.43±137.41 nM (n=8), p<0.0001).

TABLE 3
NO and H2S levels based on biomarkers and disease severity.

| | NO Levels (nM) | | | | H2S Levels (μM) | | | |
|---|---|---|---|---|---|---|---|---|
| | Mean (n) | | ±SD | p-Value | Mean (n) | | ±SD | p-Value |
| Troponin | | | | 0.14 | | | | 0.64 |
| Troponin ≤ 0.04 ng/ml | 273.87 | (30) | 137.36 | | 1.17 | (31) | 0.21 | |
| Troponin > 0.04 ng/ml | 347.54 | (13) | 176.18 | | 1.14 | (15) | 0.25 | |
| D-Dimer | | | | 0.08 | | | | 0.85 |
| D-Dimer ≤ 1000 ng/ml | 240.00 | (14) | 111.99 | | 1.14 | (15) | 0.15 | |
| D-Dimer > 1000 ng/ml | 326.45 | (31) | 163.98 | | 1.13 | (36) | 0.24 | |
| Ferritin | | | | 0.43 | | | | 0.94 |
| Ferritin ≤ 600 ng/ml | 266.12 | (17) | 141.11 | | 1.14 | (18) | 0.13 | |
| Ferritin > 600 ng/ml | 300.94 | (35) | 153.24 | | 1.14 | (40) | 0.23 | |
| CRP | | | | 0.56 | | | | 0.76 |
| CRP ≤ 1.8 mg/dl | 325.33 | (9) | 138.17 | | 1.15 | (9) | 0.21 | |
| CRP > 1.8 mg/dl | 294.62 | (37) | 141.57 | | 1.18 | (43) | 0.21 | |
| LDH | | | | 0.004 | | | | 0.22 |
| LDH ≤ 260 U/L | 210.50 | (10) | 64.41 | | 1.08 | (11) | 0.17 | |
| LDH > 260 U/L | 323.16 | (31) | 167.63 | | 1.17 | (35) | 0.22 | |
| BNP | | | | 0.21 | | | | 0.11 |
| BNP ≤ 1000 pg/ml | 255.00 | (15) | 131.61 | | 1.09 | (16) | 0.12 | |
| BNP > 1000 pg/ml | 322.53 | (17) | 163.45 | | 1.19 | (20) | 0.21 | |
| RDW | | | | 0.16 | | | | 0.92 |
| RDW ≤ 14.5% | 265.86 | (36) | 109.38 | | 1.14 | (37) | 0.23 | |
| RDW > 14.5% | 323.17 | (24) | 174.51 | | 1.15 | (30) | 0.18 | |
| Lactate | | | | 0.05 | | | | 0.10 |
| Lactate ≤ 1.25 mmol/L | 235.65 | (17) | 64.79 | | 1.08 | (17) | 0.22 | |
| Lactate > 1.25 mmol/L | 311.26 | (27) | 177.96 | | 1.19 | (31) | 0.21 | |
| Procalcitonin | | | | 0.05 | | | | 0.14 |
| Procalcitonin ≤ 0.5 ng/ml | 251.34 | (29) | 102.73 | | 1.12 | (31) | 0.23 | |
| Procalcitonin > 0.5 ng/ml | 334.70 | (20) | 163.57 | | 1.21 | (23) | 0.19 | |
| Severity of COVID | | | | 0.29 | | | | 0.009 |
| Mild-Moderate | 274.02 | (46) | 133.67 | | 1.10 | (46) | 0.17 | |
| Severe | 314.68 | (22) | 154.25 | | 1.23 | (22) | 0.24 | |
| Outcome | | | | <0.001 | | | | 0.013 |
| Alive | 263.65 | (60) | 124.34 | | 1.11 | (60) | 0.17 | |
| Expired | 464.43 | (8) | 137.41 | | 1.40 | (8) | 0.25 | |

CRP—C-Reactive Protein;
LDH—Lactate Dehydrogenase;
BNP—Brain Natriuretic Peptide;
RDW—Red cell Distribution Width.

We further analyzed the relationship between biomarkers and H₂S levels in the COVID-19 patients and found a significant increase in H₂S levels (1.10±0.17 μM versus 1.23±0.24 μM, p=0.009) in patients with severe COVID-19 illness compared to those with mild-to-moderately severe COVID-19 illness. Levels of H₂S significantly increased in expired patients compared to levels in those who survived (1.11±0.17 μM versus 1.40±0.25 μM, p<0.013). To assess if the higher NO and H₂S levels in sicker COVID-19 patients and COVID-19 patients who expired reflected a higher demand due to advanced oxidant stress, we compared nitrotyrosine levels in mild to moderately ill COVID-19 patients and patients who survived the COVID-19 infection to severely ill COVID-19 patients and COVID-19 patients who succumbed to their illness. Severely ill COVID-19 patients had significantly higher nitrotyrosine levels compared to mild to moderately ill patients (128.76±55.55 nM versus 93.51±60.95 nM, p=0.04). Similarly, patients who died from COVID-19 infection had a trend towards a higher nitrotyrosine level compared to patients who survived (139.45±59.26 nM versus 99.96±60.40 nM, p=0.11). The patients who had higher levels of cardiac, inflammatory, and thrombosis biomarkers had higher NO and H₂S levels although most were non-significant (Table 3).

We performed multivariable regression analysis to identify any association between comorbidities and total NO and sulfide levels (Table 4). It is worth noting that we did not find any further association between NO and H₂S levels and cardiovascular risk factors, including age, race, sex, diabetes, and hypertension (Table 4).

TABLE 4
Multivariable regression analysis of association of demographics
and comorbidities with total NO and sulfide levels.

| | | |
|---|---|---|
| Risk Factor | Coefficient ± SD | p-Value |

Multivariable Regression analysis in COVID positive cases-
Total Nitric Oxide

| | | |
|---|---|---|
| Age | 0.35 ± 1.45 | 0.81 |
| Race | −2.31 ± 38.76 | 0.95 |
| Gender | 51.41 ± 40.3 | 0.21 |
| Diabetes Mellitus | −32.29 ± 44.62 | 0.47 |
| Hypertension | 27.03 ± 49.1 | 0.58 |

Multivariable Regression analysis in COVID positive cases-
Total Sulfide

| | | |
|---|---|---|
| Age | 0.0 | 0.8 |
| Race | −0.02 ± 0.06 | 0.68 |
| Gender | −0.04 ± 0.06 | 0.49 |
| Diabetes Mellitus | 0.04 ± 0.06 | 0.5 |
| Hypertension | −0.07 ± 0.07 | 0.31 |

Discussion. The gasotransmitters NO and H₂S have overlapping pathophysiological roles with significant influence in regulating cardio- and vaso-protective functions and possessing anti-inflammatory, anti-thrombotic, and antiviral properties. While researchers have pondered the possible use of NO and H₂S in the treatment of COVID-19, studies exploring the availability of these two gasotransmitters in COVID-19 patients are limited. For the first time, our study analyzed and compared both NO and sulfide metabolites in healthy subjects and COVID-19 patients and observed a significant and parallel reduction in both NO and sulfide metabolites in the COVID-19 patients compared to controls (FIG. 1, FIG. 2).

NO plays a key protective role in limiting the severity of cardiovascular disease (CVD), and as a selective pulmonary vasodilator, improves pulmonary function in subjects with acute and chronic pulmonary hypertension. Previously, NO has been negatively associated with viral replication in severe acute respiratory syndrome (SARS/SARS-CoV). In vitro studies with SARS-CoV suggested to the inventors that NO has anti-viral properties as shown by its specific inhibition of the viral replication cycle. Chen et al. demonstrated the favorable effect of inhaled NO on arterial oxygenation in patients with acute respiratory distress syndrome. Similar to SARS-CoV-1, SARS-CoV-2 infects the upper respiratory tract, but with increased complications mediated through vascular inflammation and injury. It has been predicted that COVID-19 mortality could be associated with decreased endothelial NO production and availability. Based on earlier reports from studies of SARS-CoV-1, the inhibitory effect of NO on SARS-CoV-2 has been evaluated recently in vitro and found to promote significant reduction in SARS-CoV-2 protease activity. Although there are now clinical trials using NO therapy to alleviate viral pneumonia and the bronchopulmonary effects of SARS-CoV-2, interestingly, there have been no reports suggesting a decrease in NO availability in COVID-19 patients. Recently, a study by Alamdari et al. showed a significant increase in NO levels in 25 COVID-19 patients in ICU compared to non-infected controls, but did not include data from mildly ill COVID-19 patients. In contrast, our study found significantly lower NO metabolites in patients with COVID-19 infections of different severities compared to controls.

H₂S is another gasotransmitter with antiviral properties that is cardioprotective, anti-inflammatory, and antioxidant. We have previously reported H₂S availability as a predictive biomarker for cardiovascular disease in a race- and sex-based manner. A recent study has suggested a correlation between the severity of SARS-CoV-2 infection, cytokine production, and H₂S plasma level. H₂S levels were significantly reduced in deceased patients compared to those who survived following COVID-19 infection, suggesting a possible role of H₂S in the outcome of pneumonia caused by SARS-CoV-2. However, that study was limited to COVID-19 patients with viral pneumonia and did not include non-infected controls. In a biological system, H₂S can be present in various forms including free, acid labile, and bound sulfane sulfur that regulate and contribute to the total amount of bioavailable sulfide. For the first time, we demonstrate that all of these sulfide biochemical forms are significantly reduced in COVID-19 patients compared to healthy controls (FIG. 2). The interaction between H₂S and NO can be complex and could range from synergism, based on evidence from the cardiovascular disease models. to antagonistic regulation of each other found in inflammatory cells, especially in pulmonary infections. Our finding that both H₂S and NO are reduced in COVID-19 infection simultaneously hints at a more synergistic role for these two gasotransmitters in this context.

There are known variations in NO and H₂S levels based on race in vascular disease patients. ROC analyses with NO showed a significantly predictable relationship between COVID-19 and NO levels, including total NO, free nitrite, and SNO metabolites in all of the COVID-19 subjects, irrespective of race (FIGS. 6A-6C). Interestingly, sulfide metabolites, especially total sulfide and free sulfide, were more predictive of COVID-19 infection than NO metabolites. ROC analysis of free sulfide showed that a free sulfide level of 0.30 μM was 96% sensitive and 33% specific in predicting COVID-19 infection in the general population; >94% sensitive and 58% specific in the Caucasian population; and 95% sensitive and 14% specific in the AA population. Assuming a roughly 10% prevalence of COVID-19 infection in the United States, free sulfide levels of 0.30 μM predicted COVID-19 infection with a positive predictive value (PPV) of 14% but a negative predictive value (NPV) of 99% in the general population and a PPV of 20% and a NPV of 99% in Caucasians, suggesting that higher free sulfide levels can rule out COVID-19 infection with certainty. The majority of the control population in this study was healthy and did not have significant comorbidities, while 25% of COVID-19 cases had CVD. Previously, we have shown that while the levels of other sulfide metabolites in the plasma are decreased with cardiovascular disease, free sulfide levels are elevated in these patients. Therefore, the finding that free sulfide levels are significantly reduced in and are the best predictors of COVID-19 infection in the COVID-19 cases with 25% CVD prevalence assumes prominence.

Elevated levels of multiple biomarkers including lactate dehydrogenase (LDH) and procalcitonin were associated with poor outcomes in COVID-19 infection. We therefore analyzed the effects of various inflammatory and cardiovascular biomarkers on NO and H₂S in COVID-19 patients (Table 3). We saw a significant association between LDH and NO levels in COVID-19 infected subjects (Table 3). Surprisingly, patients with LDH levels >260 U/L had higher total NO levels compared to patients with LDH levels ≤260 U/L. NO also showed a significant association with mortality, with increased NO levels in expired COVID-19 subjects compared to patients who survived. This agrees with the findings in the study by Alamdari et al. Similarly, COVID-19 patients who were severely ill or expired had a significantly higher plasma H₂S levels compared to patients who were mild-to-moderately ill or survived. Although the gasotransmitter levels were significantly reduced in COVID-19 patients compared to controls, it is unclear why sicker COVID-19 patients had relatively elevated levels compared to less sick patients. One possible explanation is that the elevated NO and H₂S levels in sicker COVID-19 patients is a last-ditch compensatory response to the severely noxious effects of the COVID-19 infection. Another reason could be a hypothetical inability to utilize or underutilization of NO and H₂S to reduce oxidative stress leading to poor outcomes. NO-derived oxidant generation can also reduce NO availability, thereby reducing its levels. Peroxynitrite is one such oxidant that promotes nitration of protein tyrosine residues such as nitrotyrosine. We observed a significant increase in nitrotyrosine levels in the plasma of COVID-19 patients (FIG. 5) in conjunction with reduced NO levels. In addition, severely ill COVID-19 patients had significantly higher nitrotyrosine levels compared to mild-moderately ill COVID-19 patients and COVID-19 patients who died had a trend towards higher nitrotyrosine levels compared to COVID-19 patients who survived, lending credibility to this hypothesis. Finally, changes in circulating NO levels could reflect alterations in nitric oxide synthase (NOS). Decreased NO availability generally can be attributed to reduced eNOS; whereas iNOS is correlated with high NO production. A direct association of eNOS and iNOS, including eNOS polymorphisms have been proposed to critically regulate defense against SARS-CoV-2 and COVID-19 severity. While iNOS is likely activated by the inflammation and cytokine storm caused by COVID-19, our finding that total NO levels in patients with COVID-19 is low could suggest an overwhelming effect of COVID-19 on endothelial NOS, resulting in high oxidant stress which in turn could possibly result in NOS uncoupling. The variations in the level of iNOS between moderately and severely ill COVID-19 patients could also explain the differences in NO levels in these patient groups and the findings in our study compared to the study by Alamdari et al.

Comorbidities in COVID-19 patients may be associated with increased hospitalizations, complications, and mortality. Therefore, we used multivariable regression analyses to find the association between the gasotransmitters NO and H₂S and other risk factors (Table 3) in COVID-19 positive cases. Remarkably, there were no further differences in either NO or sulfide metabolites with patient demographics or cardiovascular comorbidities known to affect their levels, including age, race, sex, diabetes, and hypertension (Table 4), suggesting that the effect of COVID-19 on these gasotransmitters was overwhelming, leaving no room for variations.

Conclusion and future directions. In summary, our findings reveal that the availability NO and sulfide metabolites is significantly reduced in individuals with COVID-19 infection but is not affected by comorbidities. In addition, reduced free sulfide levels have a high sensitivity in predicting COVID-19 infection in the study population regardless of race. Based on a case study within the cohort, inflammatory and oxidative stress markers CRP and nitrotyrosine, were inversely related to NO/H₂S availability with the onset of COVID-19 infection. Overall, our study further substantiates the need for NO as a therapeutic modality for COVID-19, consistent with ongoing clinical trials. Additionally, our study also provided evidence for exogenous H₂S therapy as a pharmacological strategy, especially for mild to moderate COVID-19 disease, to restore its availability and counteract the severe consequences of COVID-19 infection. Finally, based on this association of decreasing NO and H₂S availability with COVID-19 infection, it is evidenced that these gasotransmitters are protective factors and novel therapeutic alternatives.

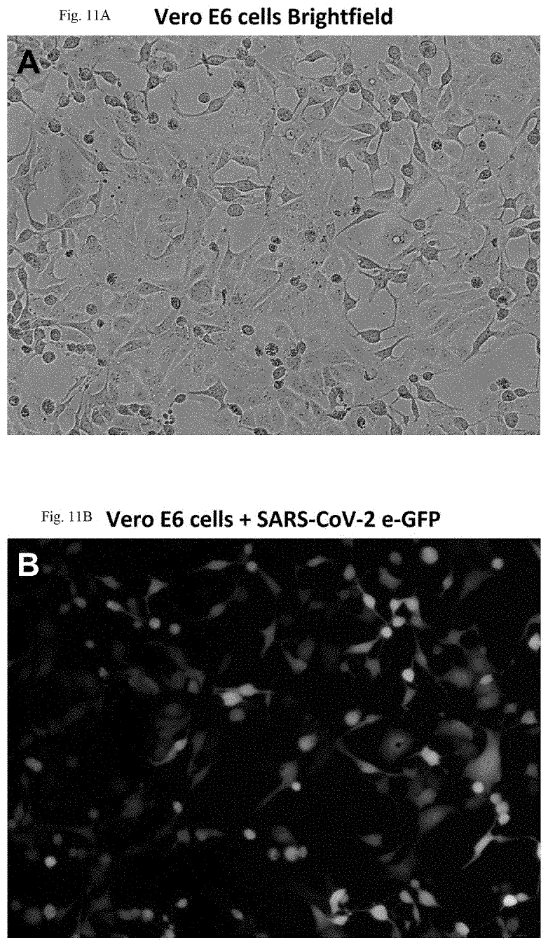

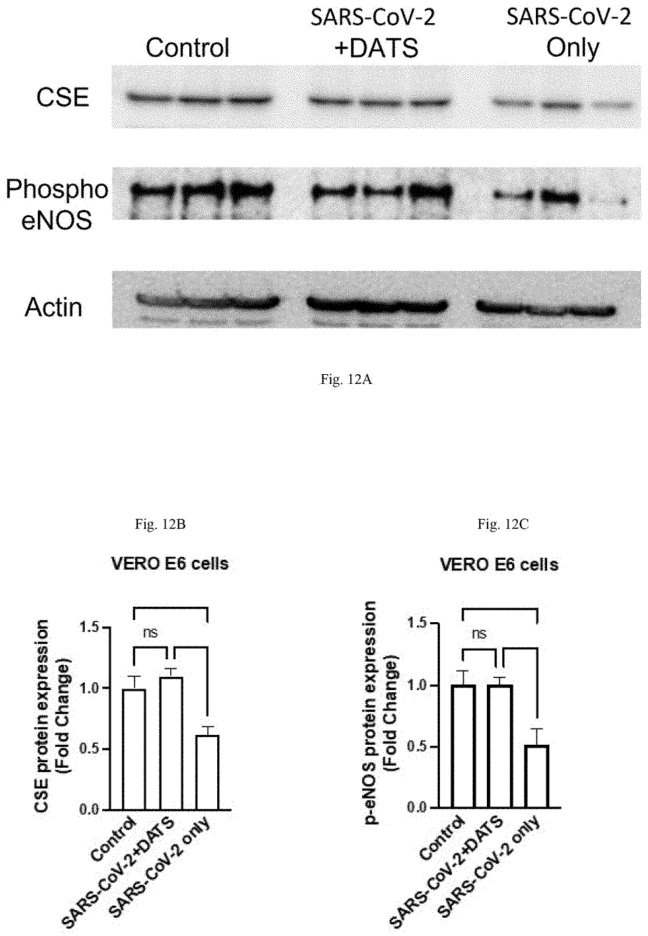

Further proof of concept experimentation conducted. We observed through our experiments above that H₂S and NO are significantly reduced in COVID-19 patients irrespective of their comorbidities. They act as biomarkers for COVID-19 on severity basis. We have also showed that H₂S can increase NO bioavailability by inducing eNOS activity via its phosphorylation at Ser1177 site (p-eNOS). In the following data (as shown in FIGS. 11 and 12) we used an infectious VSV Chimera with SARS-CoV-2 S Protein to generate a replication-competent virus to study entry and neutralization of VSV-eGFPSARS-CoV-2-SAA (simply SARS-CoV-2) at our BSL2 facility (FIG. 11B). We treated the kidney epithelial cells (Vero E6) with the eGFP tagged virus to identify protein expression changes in H₂S producing enzyme, CSE and active form of eNOS, p-eNOS that produces NO. We observed a significant reduction in both CSE and p-eNOS in VERO e6 cells when infected with SARS-CoV2 for 48 hrs. Interestingly, co-treatment with H₂S donor, 50 mM DATS significantly restored both CSE and p-eNOS protein expressions by two-fold (FIG. 12).

These results indicate that H2S therapy significantly restores SARS-CoV-2 effected H2S and NO signaling at cellular level by inhibiting CSE and p-eNOS protein expressions. This also indicates that H₂S-based drugs can are an effective therapeutic agent to restore cellular signaling and function at cellular and organ level in cells and organs infected with SARS-CoV-2.

Pharmaceutical Compositions. The methods described herein can also include the administrations of pharmaceutically acceptable compositions that include the therapeutic, or a pharmaceutically acceptable salt, solvate, or prodrug thereof. When employed as pharmaceuticals, any of the present compounds can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical, parenteral, intravenous, intra-arterial, subcutaneous, intramuscular, intracranial, intraorbital, ophthalmic, intraventricular, intracapsular, intraspinal, intracisternal, intraperitoneal, intranasal, aerosol, by suppositories, or oral administration.

This invention also includes pharmaceutical compositions which can contain one or more pharmaceutically acceptable carriers. In making the pharmaceutical compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semisolid, or liquid material (e.g., normal saline), which acts as a vehicle, carrier or medium for the active ingredient. Thus, the compositions can be in the form of tablets, powders, lozenges, sachets, cachets, elixirs, suspensions, emulsions, solutions, syrups, and soft and hard gelatin capsules. As is known in the art, the type of diluent can vary depending upon the intended route of administration. The resulting compositions can include additional agents, such as preservatives.

The therapeutic agents of the invention can be administered alone, or in a mixture, in the presence of a pharmaceutically acceptable excipient or carrier. The excipient or carrier is selected on the basis of the mode and route of administration. Suitable pharmaceutical carriers, as well as pharmaceutical necessities for use in pharmaceutical formulations, are described in Remington: The Science and Practice of Pharmacy, 22^{nd} Ed., Gennaro, Ed., Lippencott Williams & Wilkins (2012), a well-known reference text in this field, and in the USP/NF (United States Pharmacopeia and the National Formulary), each of which is incorporated by reference. In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

Examples of suitable excipients are lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. Other exemplary excipients are described in Handbook of Pharmaceutical Excipients, 8^{th} Edition, Sheskey et al., Eds., Pharmaceutical Press (2017), which is incorporated by reference.

The methods described herein can include the administration of a therapeutic, or prodrugs or pharmaceutical compositions thereof, or other therapeutic agents. Exemplary therapeutics include those that increase patient H2S concentration (including H2S donors, such as DATS and Sugammadex, for example) and increase patient NO concentration (including nitrites, like inorganic nitrites, such as NaNO2 for example).

The pharmaceutical compositions can be formulated so as to provide immediate, extended, or delayed release of the active ingredient after administration to the patient by employing procedures known in the art.

The compositions can be formulated in a unit dosage form, each dosage containing, e.g., 0.1-500 mg of the active ingredient. For example, the dosages can contain from about 0.1 mg to about 50 mg, from about 0.1 mg to about 40 mg, from about 0.1 mg to about 20 mg, from about 0.1 mg to about 10 mg, from about 0.2 mg to about 20 mg, from about 0.3 mg to about 15 mg, from about 0.4 mg to about 10 mg, from about 0.5 mg to about 1 mg; from about 0.5 mg to about 100 mg, from about 0.5 mg to about 50 mg, from about 0.5 mg to about 30 mg, from about 0.5 mg to about 20 mg, from about 0.5 mg to about 10 mg, from about 0.5 mg to about 5 mg; from about 1 mg from to about 50 mg, from about 1 mg to about 30 mg, from about 1 mg to about 20 mg, from about 1 mg to about 10 mg, from about 1 mg to about 5 mg; from about 5 mg to about 50 mg, from about 5 mg to about 20 mg, from about 5 mg to about 10 mg; from about 10 mg to about 100 mg, from about 20 mg to about 200 mg, from about 30 mg to about 150 mg, from about 40 mg to about 100 mg, from about 50 mg to about 100 mg of the active ingredient, from about 50 mg to about 300 mg, from about 50 mg to about 250 mg, from about 100 mg to about 300 mg, or, from about 100 mg to about 250 mg of the active ingredient. For preparing solid compositions such as tablets, the principal active ingredient is mixed with one or more pharmaceutical excipients to form a solid bulk formulation composition containing a homogeneous mixture of a compound of the present invention. When referring to these bulk formulation compositions as homogeneous, the active ingredient is typically dispersed evenly throughout the composition so that the composition can be readily subdivided into equally effective unit dosage forms such as tablets and capsules. This solid bulk formulation is then subdivided into unit dosage forms of the type described above containing from, for example, 0.1 to about 500 mg of the active ingredient of the present invention.

Compositions for Oral Administration. The pharmaceutical compositions contemplated by the invention include those formulated for oral administration (“oral dosage forms”). Oral dosage forms can be, for example, in the form of tablets, capsules, a liquid solution or suspension, a powder, or liquid or solid crystals, which contain the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Formulations for oral administration may also be presented as chewable tablets, as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Controlled release compositions for oral use may be constructed to release the active drug by controlling the dissolution and/or the diffusion of the active drug substance. Any of a number of strategies can be pursued in order to obtain controlled release and the targeted plasma concentration vs time profile. In one example, controlled release is obtained by appropriate selection of various formulation parameters and ingredients, including, e.g., various types of controlled release compositions and coatings. Thus, the drug is formulated with appropriate excipients into a pharmaceutical composition that, upon administration, releases the drug in a controlled manner. Examples include single or multiple unit tablet or capsule compositions, oil solutions, suspensions, emulsions, microcapsules, microspheres, nanoparticles, patches, and liposomes. In certain embodiments, compositions include biodegradable, pH, and/or temperature-sensitive polymer coatings.

Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

The liquid forms in which the compounds and compositions of the present invention can be incorporated for administration orally include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions suitable for oral mucosal administration (e.g., buccal or sublingual administration) include tablets, lozenges, and pastilles, where the active ingredient is formulated with a carrier, such as sugar, acacia, tragacanth, or gelatin and glycerine.

Coatings. The pharmaceutical compositions formulated for oral delivery, such as tablets or capsules of the present invention can be coated or otherwise compounded to provide a dosage form affording the advantage of delayed or extended release. The coating may be adapted to release the active drug substance in a predetermined pattern (e.g., in order to achieve a controlled release formulation) or it may be adapted not to release the active drug substance until after passage of the stomach, e.g., by use of an enteric coating (e.g., polymers that are pH-sensitive (“pH controlled release”), polymers with a slow or pH-dependent rate of swelling, dissolution or erosion (“time-controlled release”), polymers that are degraded by enzymes (“enzyme-controlled release” or “biodegradable release”) and polymers that form firm layers that are destroyed by an increase in pressure (“pressure-controlled release”)). Exemplary enteric coatings that can be used in the pharmaceutical compositions described herein include sugar coatings, film coatings (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or coatings based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose. Furthermore, a time delay material such as, for example, glyceryl monostearate or glyceryl distearate, may be employed.

For example, the tablet or capsule can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release.

When an enteric coating is used, desirably, a substantial amount of the drug is released in the lower gastrointestinal tract.

In addition to coatings that effect delayed or extended release, the solid tablet compositions may include a coating adapted to protect the composition from unwanted chemical changes (e.g., chemical degradation prior to the release of the active drug substance). The coating may be applied on the solid dosage form in a similar manner as that described in Encyclopedia of Pharmaceutical Technology, vols. 5 and 6, Eds. Swarbrick and Boyland, 2000.

Parenteral Administration. Within the scope of the present invention are also parenteral depot systems from biodegradable polymers. These systems are injected or implanted into the muscle or subcutaneous tissue and release the incorporated drug over extended periods of time, ranging from several days to several months. Both the characteristics of the polymer and the structure of the device can control the release kinetics which can be either continuous or pulsatile. Polymer-based parenteral depot systems can be classified as implants or microparticles. The former are cylindrical devices injected into the subcutaneous tissue whereas the latter are defined as spherical particles in the range of 10-100 μm. Extrusion, compression or injection molding are used to manufacture implants whereas for microparticles, the phase separation method, the spray-drying technique and the water-in-oil-in-water emulsion techniques are frequently employed. The most commonly used biodegradable polymers to form microparticles are polyesters from lactic and/or glycolic acid, e.g. poly(glycolic acid) and poly(L-lactic acid) (PLG/PLA microspheres). Of particular interest are in situ forming depot systems, such as thermoplastic pastes and gelling systems formed by solidification, by cooling, or due to the sol-gel transition, cross-linking systems and organogels formed by amphiphilic lipids. Examples of thermosensitive polymers used in the aforementioned systems include, N-isopropylacrylamide, poloxamers (ethylene oxide and propylene oxide block copolymers, such as poloxamer 188 and 407), poly(N-vinyl caprolactam), poly(siloethylene glycol), polyphosphazenes derivatives and PLGA-PEG-PLGA.

Mucosal Drug Delivery. Mucosal drug delivery (e.g., drug delivery via the mucosal linings of the nasal, rectal, vaginal, ocular, or oral cavities) can also be used in the methods described herein. Methods for oral mucosal drug delivery include sublingual administration (via mucosal membranes lining the floor of the mouth), buccal administration (via mucosal membranes lining the cheeks), and local delivery (Harris et al., Journal of Pharmaceutical Sciences, 81(1): 1-10, 1992).

Oral transmucosal absorption is generally rapid because of the rich vascular supply to the mucosa and allows for a rapid rise in blood concentrations of the therapeutic.

For buccal administration, the compositions may take the form of, e.g., tablets, lozenges, etc. formulated in a conventional manner. Permeation enhancers can also be used in buccal drug delivery. Exemplary enhancers include 23-lauryl ether, aprotinin, azone, benzalkonium chloride, cetylpyridinium chloride, cetyltrimethylammonium bromide, cyclodextrin, dextran sulfate, lauric acid, lysophosphatidylcholine, methol, methoxysalicylate, methyloleate, oleic acid, phosphatidylcholine, polyoxyethylene, polysorbate 80, sodium EDTA, sodium glycholate, sodium glycodeoxycholate, sodium lauryl sulfate, sodium salicylate, sodium taurocholate, sodium taurodeoxycholate, sulfoxides, and alkyl glycosides. Bioadhesive polymers have extensively been employed in buccal drug delivery systems and include cyanoacrylate, polyacrylic acid, hydroxypropyl methylcellulose, and poly methacrylate polymers, as well as hyaluronic acid and chitosan.

Liquid drug formulations (e.g., suitable for use with nebulizers and liquid spray devices and electrohydrodynamic (EHD) aerosol devices) can also be used. Other methods of formulating liquid drug solutions or suspension suitable for use in aerosol devices are known to those of skill in the art (see, e.g., Biesalski, U.S. Pat. No. 5,112,598, and Biesalski, U.S. Pat. No. 5,556,611).

Formulations for sublingual administration can also be used, including powders and aerosol formulations. Exemplary formulations include rapidly disintegrating tablets and liquid-filled soft gelatin capsules.

Dosing Regimes. The present methods for treating COVID-19s are carried out by administering a therapeutic for a time and in an amount sufficient to result in decreased conditions or symptoms of the infection.

The amount and frequency of administration of the compositions can vary depending on, for example, what is being administered, the state of the patient, and the manner of administration. In therapeutic applications, compositions can be administered to a patient suffering from COVID-19 in an amount sufficient to relieve or least partially relieve the symptoms of COVID-19 and its complications. The dosage is likely to depend on such variables as the type and extent of progression of COVID-19, the severity of COVID-19, the age, weight and general condition of the particular patient, the relative biological efficacy of the composition selected, formulation of the excipient, the route of administration, and the judgment of the attending clinician. Effective doses can be extrapolated from dose-response curves derived from in vitro or animal model test system. An effective dose is a dose that produces a desirable clinical outcome by, for example, improving a sign or symptom of COVID-19 or slowing its progression.

The amount of therapeutic per dose can vary. For example, a subject can receive from about 0.1 μg/kg to about 10,000 μg/kg. Generally, the therapeutic is administered in an amount such that the peak plasma concentration ranges from 150 nM-250 μM.

Exemplary dosage amounts can fall between 0.1-5000 μg/kg, 100-1500 μg/kg, 100-350 μg/kg, 340-750 μg/kg, or 750-1000 μg/kg. Exemplary dosages can 0.25, 0.5, 0.75, 1°, or 2 mg/kg. In another embodiment, the administered dosage can range from 0.05-5 mmol of therapeutic (e.g., 0.089-3.9 mmol) or 0.1-50 μmol of therapeutic (e.g., 0.1-25 μmol or 0.4-20 μmol).

The plasma concentration of therapeutic can also be measured according to methods known in the art. Exemplary peak plasma concentrations of therapeutic can range from 0.05-10 μM, 0.1-10 μM, 0.1-5.0 μM, or 0.1-1 μM. Alternatively, the average plasma levels of therapeutic can range from 400-1200 μM (e.g., between 500-1000 μM) or between 50-250 μM (e.g., between 40-200 μM). In some embodiments where sustained release of the drug is desirable, the peak plasma concentrations (e.g., of therapeutic) may be maintained for 6-14 hours, e.g., for 6-12 or 6-10 hours. In other embodiments where immediate release of the drug is desirable, the peak plasma concentration (e.g., of therapeutic) may be maintained for, e.g., 30 minutes.

The frequency of treatment may also vary. The subject can be treated one or more times per day with therapeutic (e.g., once, twice, three, four or more times) or every so-many hours (e.g., about every 2, 4, 6, 8, 12, or 24 hours). Preferably, the pharmaceutical composition is administered 1 or 2 times per 24 hours. The time course of treatment may be of varying duration, e.g., for two, three, four, five, six, seven, eight, nine, ten or more days. For example, the treatment can be twice a day for three days, twice a day for seven days, twice a day for ten days. Treatment cycles can be repeated at intervals, for example weekly, bimonthly or monthly, which are separated by periods in which no treatment is given. The treatment can be a single treatment or can last as long as the life span of the subject (e.g., many years).

PREFERRED DOSING REGIMES. Patients with COVID-19 may be treated with a sodium nitrite, preferably 10 mg to 100 mg, more preferably 20 mg to 70 mg, most preferably 40 mg, orally twice daily for a 3-month period to not only combat the acute COVID-19 illness but to also reduce the proportion of COVID long haulers. If patients are acutely sick and are intubated a first dose of sodium nitrite at preferably 40 mg to 120 mg, more preferably 50 mg to 80 mg, and most preferably 60 mg will be given as an intravenous infusion followed by preferably 20 mg to 80 mg, more preferably 30 mg to 60 mg, and most preferably 40 mg orally twice daily or through the NG tube. If patients are not able to take anything through the NG tube due to gut motility issues or other GI issues, preferably 40 mg to 120 mg, more preferably 50 mg to 80 mg, and most preferably 60 mg IV once a day will be continued until the patient can take the drug orally. As one of the goals is reduce COVID long haulers and therefore a routine 3-month administration is preferably be carried out. If patients continue to have symptoms of fatigue, loss of smell, respiratory distress, and objective findings of depressed left ventricular systolic function on ECHO, EKG abnormalities or increased D-dimer or CRP on blood work continued beyond 3 months, the dosage will preferably be extended until resolution of the symptoms, signs or lab tests. In selected patients with severe respiratory illness preferably 40 mg to 160 mg, more preferably 60 mg to 120 mg, and most preferably 80 mgs of sodium nitrite will be used in a micro-nebulized route three times a day for 3 weeks with or without oral nitrite therapy. Finally, nitrite therapy may or may not be combined with sulfide therapy in the COVID-19 patients. In some patients where nitrite therapy may not be tolerated due to hypotension or is contraindicated, treatment with sulfide alone will be administered. Sulfide donor sodium sulfide will be used at preferably 0.20 mg/kg/hr to 3.00 mg/kg/hr, more preferably 0.50 mg/kg/hr to 1.50 mg/kg/hr, and most preferably 0.75 mg/kg/hr as a continuous infusion if the patients are admitted to the hospital as early as possible after the admission. Alternately, we will give a single bolus dose of a sulfide donor, such as Sugammadex, at a dosage range of preferably 0.05 mg/kg to 3.00 mg/kg, more preferably 1.00 mg/kg to 1.50 mg/kg, and most preferably 0.75 mg/kg. Intubated patients may also receive supplemental gaseous form of disodium sulfide at preferably 10.0 ppm to 150.0 ppm, more preferably from 30 ppm to 120 ppm, and most preferably 60 ppm. For outpatients and long-term maintenance preferably 10 mg/kg sodium sulfide will be administered once or twice a day for a 3 month period or until resolution of symptoms or reversal of lab abnormalities or other imaging abnormalities, whichever is longer.

Kits. Any of the pharmaceutical compositions of the invention described herein can be used together with a set of instructions, i.e., to form a kit. The kit may include instructions for use of the pharmaceutical compositions as a therapy as described herein. For example, the instructions may provide dosing and therapeutic regimes for use of the compounds of the invention to reduce symptoms and/or underlying cause of the COVID-19 infection.

The invention illustratively disclosed herein suitably may explicitly be practiced in the absence of any element which is not specifically disclosed herein. While various embodiments of the present invention have been described in detail, it is apparent that various modifications and alterations of those embodiments will occur to and be readily apparent those skilled in the art. However, it is to be expressly understood that such modifications and alterations are within the scope and spirit of the present invention, as set forth in the appended claims. Further, the invention(s) described herein is capable of other embodiments and of being practiced or of being carried out in various other related ways. The present disclosure also contemplates other embodiments “comprising,” “consisting of” and “consisting essentially of,” the embodiments or elements presented herein, whether explicitly set forth or not. In addition, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items, while only the terms “consisting of” and “consisting only of” are to be construed in the limitative sense. cm Wherefore, I/We claim:

1. A method of treating COVID-19 comprising in a patient comprising:
administering to the patient an effective dose of a pharmacologic composition containing a first therapeutic;
wherein the first therapeutic is a hydrogen sulfide (H2S) donor, or a salt, solvate, ester, amide, clathrate, stereoisomer, enantiomer, prodrug or analogs thereof.

2. The method of claim 1 wherein the H2S donor is one of include diallyl trisulfide (DATS), diallyl disulfide (DADS), sodium sulfide, acillin, sugammadex, sulfanilamide, disulfram, sulfonamide, a sulfinate, a sulfoxide, a persulfide, a polysulfide, and a sulfone.

3. The method of claim 1 wherein the pharmacologic composition further contains a second therapeutic, wherein the second therapeutic is a nitrite, or a salt, solvate, ester, amide, clathrate, stereoisomer, enantiomer, prodrug or analogs thereof.

4. The method of claim 3, wherein the nitrite is an inorganic nitrite.

5. The method of claim 4, wherein the inorganic nitrite is one of sodium nitrite (NaNO₂), ammonium nitrite (NH₄NO₂), barium nitrite (Ba(NO₂)₂; e.g., anhydrous barium nitrite or barium nitrite monohydrate), calcium nitrite (Ca(NO₂)₂; e.g., anhydrous calcium nitrite or calcium nitrite monohydrate), cesium nitrite (CsNO₂), cobalt(II)nitrite (Co(NO₂)₂), cobalt(III)potassium nitrite (CoK₃(NO₂)₆; e.g., cobalt(III)potassium nitrite sesquihydrate), lithium nitrite (LiNO₂; e.g., anhydrous lithium nitrite or lithium nitrite monohydrate), magnesium nitrite (MgNO₂; e.g., magnesium nitrite trihydrate), potassium nitrite (KNO₂), rubidium nitrite (RbNO₂), silver(I)nitrite (AgNO₂), strontium nitrite (Sr(NO₂)₂), and zinc nitrite (Zn(NO₂)₂).

6. The method of claim 5, wherein the inorganic nitrite is NaNO₂.

7. A method of diagnosing a severity of a COVID-19 infection in a patient comprising:
measuring a level of one, two of, or all three of NO metabolites, sulfide metabolites, and nitrotyrosine in a patient;
diagnosing the patient with having a severe COVID-19 infection if one of, two of, or all three of the NO metabolite level is low compared to a normal NO metabolite level, the sulfide metabolite level is low compared to a normal sulfide metabolite level, and the nitrotyrosine level is high compared to a normal nitrotyrosine level.

8. The method of claim 7 wherein a plasma sample from the patient is used to test the one of, two of, or all three of the NO metabolite, sulfide metabolite, and nitrotyrosine.

9. The method of claim 7 wherein the sulfide metabolite level includes one, two, or all three of a plasma free sulfide level, an acid labile sulfide level, and a total sulfide level.

10. The method of claim 9, wherein the sulfide metabolite level is not singularly a bound sulfane sulfur level.

11. The method of claim 7 wherein the nitrite metabolite level includes one, two, or all three of a total NO level, a free nitrite level and a S-nitrosothiol (SNO) level.

12. The method of claim 7 wherein a respective level is considered low if it is one of 10% lower, 20% lower, 30% lower, 40% lower, and 50% lower than a respective the normal level, and is considered high if it is one of 10% higher, 20% higher, 30% higher, 40% higher, and 50% higher than the respective normal level.

13. The method of claim 7, further comprising the step of administering to the patient an effective dose of a pharmacologic composition containing a first therapeutic, wherein the first therapeutic is a hydrogen sulfide (H2S) donor, or a salt, solvate, ester, amide, clathrate, stereoisomer, enantiomer, prodrug or analogs thereof, when the patient is diagnosed with having a severe COVID-19 infection.

14. The method of claim 13 wherein the H2S donor is one of include diallyl trisulfide (DATS), diallyl disulfide (DADS), sodium sulfide, acillin, sugammadex, sulfanilamide, disulfram, sulfonamide, a sulfinate, a sulfoxide, a persulfide, a polysulfide, and a sulfone.

15. The method of claim 14, wherein the H2S donor is sugammadex.

16. The method of claim 13 wherein the pharmacologic composition further contains a second therapeutic, wherein the second therapeutic is a nitrite, or a salt, solvate, ester, amide, clathrate, stereoisomer, enantiomer, prodrug or analogs thereof.

17. The method of claim 16, wherein the nitrite is an inorganic nitrite.

18. The method of claim 17, wherein the inorganic nitrite is one of sodium nitrite (NaNO₂), ammonium nitrite (NH₄NO₂), barium nitrite (Ba(NO₂)₂; e.g., anhydrous barium nitrite or barium nitrite monohydrate), calcium nitrite (Ca(NO₂)₂; e.g., anhydrous calcium nitrite or calcium nitrite monohydrate), cesium nitrite (CsNO₂), cobalt(II)nitrite (Co(NO₂)₂), cobalt(III)potassium nitrite (CoK₃(NO₂)₆; e.g., cobalt(III)potassium nitrite sesquihydrate), lithium nitrite (LiNO₂; e.g., anhydrous lithium nitrite or lithium nitrite monohydrate), magnesium nitrite (MgNO₂; e.g., magnesium nitrite trihydrate), potassium nitrite (KNO₂), rubidium nitrite (RbNO₂), silver(I)nitrite (AgNO₂), strontium nitrite (Sr(NO₂)₂), and zinc nitrite (Zn(NO₂)₂).

19. A method of testing for absence of active COVID-19 infection in a mammal comprising:
measuring a level of sulfide metabolite, and
determining an absence of COVID-19 infection in the mammal when the level of sulfide metabolite is equal to or greater than a normal level.

20. The method of claim 19 wherein the sulfide metabolite is free sulfide.
